(19) United States
(12) Patent Application Publication
(10) Pub. No.: US 2011/0177597 A1
(43) Pub. Date: Jul. 21, 2011

(54) CELLULAR DIFFERENTIATION PROCESS AND ITS USE FOR BLOOD VESSEL BUILD-UP

(75) Inventors: Patrick Menu, Vandoevre (FR); Nicolas Berthelemy, Nancy (FR); Halima-Assia Kerdjoudj, Vandoeuvreles Nancy (FR); Jean-Francois Stoltz, Vandoeuvreles Nancy (FR)

(73) Assignee: UNIVERSITE HENRI POINCARE NANCY 1, NANCY CEDEX (FR)

(21) Appl. No.: 13/000,928

(22) PCT Filed: Jun. 23, 2009
(86) PCT No.: PCT/EP2009/057850

§ 371 Date: Apr. 4, 2011

(30) Foreign Application Priority Data
Jun. 24, 2008 (FR) 08290599.3

(51) Int. Cl. C12N 5/071; C12N 5/077

(52) U.S. Cl. 435/377; 435/404

(57) ABSTRACT

A process of differentiation of stem cells through the use of specific oxygen concentrations, provided that the stem cells are not human embryonic stem cells, and seeded on a support, in an appropriate culture medium, wherein the differentiation leads to:
a. either a first group of specialized differentiated cells under normoxic conditions, and in an appropriate culture medium,
b. or a second group of specialized differentiated cells under hypoxic conditions, in a culture medium of the same nature as the one used for obtaining the first group of specialized differentiated cells; the first and second groups of specialized differentiated cells retaining the functional properties of the corresponding specialized differentiated cells respectively obtained through a biological natural process.

The present invention relates to a cellular differentiation process and its use for blood vessel build-up. The present invention also relates to the use of specific oxygen concentrations for the implementation of a cellular differentiation process.

In developmental biology, cellular differentiation is the process by which a less specialized cell becomes a more specialized cell type. Differentiation occurs numerous times during the development of a multicellular organism as the organism changes from a single zygote to a complex system of tissues and cell types. Differentiation is a common process in adults as well: adult stem cells divide and create fully-differentiated daughter cells during tissue repair and during normal cell turnover. Cell differentiation causes its size, shape, polarity, metabolic activity, and responsiveness to signals to change dramatically. These changes are largely due to highly-controlled modifications in gene expression. With a few exceptions, cellular differentiation almost never involves a change in the DNA sequence itself. Thus, different cells can have very different physical characteristics despite having the same genome.

A cell that is able to differentiate into many cell types is known as pluripotent. These cells are called stem cells in animals. A cell that is able to differentiate into all cell types is known as totipotent. In mammals, only the zygote and early embryonic cells are totipotent.

Development begins when a sperm fertilizes an egg and creates a single cell that has the potential to form an entire organism. In the first hours after fertilization, this cell divides into identical cells. In humans, approximately four days after fertilization and after several cycles of cell division, these cells begin to specialize, forming a hollow sphere of cells, called a blastocyst. The blastocyst has an outer layer of cells, and inside this hollow sphere, there is a cluster of cells called the inner cell mass. The cells of the inner cell mass will go on to form virtually all of the tissues of the human body. Although the cells of the inner cell mass can form virtually every type of cell found in the human body, they cannot form an organism. These cells are referred to as pluripotent.

Pluripotent stem cells undergo further specialization into multipotent progenitor cells that then give rise to functional cells. Examples of stem and progenitor cells include:
Hematopoietic stem cells (adult stem cells) from the bone marrow that give rise to red blood cells, white blood cells, and platelets
Mesenchymal stem cells (adult stem cells) from the bone marrow that give rise to stromal cells, fat cells, and types of bone cells
Epithelial stem cells (progenitor cells) that give rise to the various types of skin cells
Muscle satellite cells (progenitor cells) that contribute to differentiated muscle tissue

Each specialized cell type in an organism expresses a subset of all the genes that constitute the genome of that species. Each cell type is defined by its particular pattern of regulated gene expression. Cell differentiation is thus a transition of a cell from one cell type to another and it involves a switch from one pattern of gene expression to another. A few evolutionarily conserved types of molecular processes are often involved in the cellular mechanisms that control these switches. The major types of molecular processes that control cellular differentiation involve cell signaling. Many of the signal molecules that convey information from cell to cell during the control of cellular differentiation are called growth factors. Another important strategy is to unequally distribute molecular differentiation control signals inside a parent cell. Upon cytokinesis, the amount of such intracellular differentiation control signals can be unequal in the daughter cells and this imbalance results in distinct patterns of differentiation for the different daughter cells. A well-studied example is the body axis patterning in Drosophila. RNA molecules are an important type of intracellular differentiation control signal.

The in vitro expansion, i.e. proliferation, and differentiation processes are well documented in the art. In particular hematopoietic stem cells proliferation culture conditions for the enrichment of hematopoietic stem cells are well known.

For example, WO 2007/049096 discloses a method for expending and allowing the differentiation from hematopoietic stem cells toward endothelial cells. This method comprises an in vitro culture of stem cells, in a specific culture medium, wherein stem cells are attached on a support allowing/enhancing their differentiation into endothelial cells.

Moreover, this document never mentions that stem cells purified with the CD34-positive antigen can provide other attached cells than endothelial cells.

So, although differentiation processes are more and more understood by scientist, the mechanisms of cellular differentiation and fate remain to be elucidated.

Moreover, no document in the art discloses either method, or use of specific conditions, that allows the differentiation from stem cells toward differentiated cells, wherein said differentiated cells do not derive from said stem cells in a natural biological process.

There is a need to provide a simplest, unique or quasi unique protocol to differentiate stem cells into all wanted differentiated cells.

This need is particularly important for the surgery and the treatment of pathologies associated with either an alteration of the differentiation process, or for the organ reconstruction after an injury.

In particular, it is important to provide engineered tissues, such as blood vessels, to treat individuals with cardiovascular diseases, or vascular sickness such as emboli, vascular accident . . . for example.

All the blood vessels have the same basic structure. There are three layers, from inside to outside:
Tunica intimal (the thinnest layer): a single layer of simple squamous endothelial cells glued by a polysaccharide intercellular matrix, surrounded by a thin layer of subendothelial connective tissue interlaced with a number of circularly arranged elastic bands called the internal elastic lamina.
Tunica media (the thickest layer): circularly arranged elastic fiber, connective tissue, polysaccharide substances, the second and third layers are separated by another thick elastic band called external elastic lamina. The tunica media may (especially in arteries) be rich in vascular smooth muscle, which controls the caliber of the vessel.
Tunica adventitia: entirely made of connective tissue. It also contains nerves that supply the muscular layer, as well as nutrient capillaries (vasa vasorum) in the larger blood vessels.

The prior art discloses some processes for producing in vitro blood vessels.

WO 2005/003317 discloses a method for the in vitro build-up of a blood vessel using differentiated smooth muscle cells and endothelial cells. Moreover, this document also discloses the in vitro build-up of a blood vessel by using stem cells (or progenitor) of smooth muscle cells and of endothelial cells.

This document also discloses a matrix allowing the formation of a functional, transplantable, “engineered” blood vessel.

In the method of this document, although it is disclosed that the blood vessel is transplantable, it is needed to collect two types of stem cells for the construction of blood vessel. So the disadvantage of this method is to practice an important invasive surgery to collect usable cells.

WO 2006/099372 discloses a process for producing a blood vessel by using a matrix allowing the attachment of saphenous vein purified endothelial cells, or purified endothelial stem cells. The process disclosed in this document allows the formation of a tubular matrix wherein endothelial cells are seeded to build a vessel.

However, this document stays silent about the translatability of the in vitro produced blood vessel.

L'Heureux et al. discloses in two documents [FASEB journal, vol 12, pp 47-56 (1998); FASEB journal, vol 15, pp 515-524 (2001)] a method for producing in vitro blood vessel by using endothelial cells and smooth muscle cells isolated from umbilical cords of healthy newborn donors. In these documents, the authors disclose the production of a functional blood vessel, which is able to have contractibility features.

More recently, l'Heureux et al. [Nat. Med., 12(3) March, pp 361-364 (2006)] discloses the use of skin derived fibroblast for the formation of a support wherein smooth muscle cells and endothelial cells are able to attach, to form a new blood vessel.

The methods disclosed in these three documents allow the in vitro use of the engineered blood vessel, but, due to the origin of the used cells, dramatically reduce the possibility to transplant said engineered blood vessel and enhance the possibility of graft rejection.

The present invention provides a unique, easy to use, and rapid process to differentiate a single stem cell.

The present invention also provides a culture medium for the differentiation of stem cells, and that gives, according to the conditions, different differentiated stem cells.

The present invention also provides a process of preparation of a blood vessel using a unique type of stem cell. Said blood vessel is functional and is easily transplantable to the individual that has provided stem cell, without graft rejection.

The invention relates to the use of specific oxygen concentrations for implementing an in vitro process of differentiation of stem cells derived from bone marrow or blood or adipose tissue, or umbilical cord, provided that said stem cells are not human embryonic stem cells, and seeded on a support, in an appropriate culture medium, wherein said differentiation leads to:
a first group of specialized differentiated cells under normoxic conditions, and in an appropriate culture medium, and
or a second group of specialized differentiated cells under hypoxic conditions, in a culture medium of the same nature as the one used for obtaining the first group of specialized differentiated cells, wherein hypoxic conditions are different from anoxia,
   said first and second groups of specialized differentiated cells retaining the functional properties of the corresponding specialized differentiated cells respectively obtained through a biological natural process,
   the specialized differentiated cells of the first group having cellular functional properties different from the specialized differentiated cells of the second group.

The invention relates to the use of specific oxygen concentrations for implementing a process of differentiation, preferably in vitro, of stem cells derived from bone marrow or blood or adipose tissue, or umbilical cord, provided that said stem cells are not human embryonic stem cells, and seeded on a support, in an appropriate culture medium, wherein said differentiation leads to:
either a first group of specialized differentiated cells under normoxic conditions, and in an appropriate culture medium,
or a second group of specialized differentiated cells under hypoxic conditions, in a culture medium of the same nature as the one used for obtaining the first group of specialized differentiated cells,
   said first and second groups of specialized differentiated cells retaining the functional properties of the corresponding specialized differentiated cells respectively obtained through a biological natural process.

The present invention results from the unexpected observation that stem cells, when seeded on a support, described hereafter, in a culture medium allowing their proliferation, can differentiate in two different differentiated cells depending of the specific medium oxygen concentrations.

By “differentiation” the invention discloses the process that consists in “transforming” an immature cell to many different mature cells.

The cellular differentiation is the process by which a less specialized cell becomes a more specialized cell type.

Cell fate determination is the programming of a cell to follow a specified path of cell differentiation. Often, cells are discussed in terms of their terminal differentiation state. During development, fates of some few cells may be specified at certain times. When referring to developmental fate or cell fate, one is talking about everything that happens to that cell and its progeny after that point in development.

The process of a cell to be committed to a certain state can be divided into two stages: specification and determination. Specification is not a permanent stage and cells can be reversed based upon different cues. In contrast, determination refers to when cells are irreversibly committed to a particular fate. This is a process influenced by the action of the extracellular environment and the contents of the genome of cell. Determination is not something that is visible under the microscope cells do not change their appearance when they become determined. Determination is followed by differentiation, the actual changes in biochemistry, structure, and function that result in cells of different types. Differentiation often involves a change in appearance as well as function.

The state of commitment of a cell is also known as its developmental potential. When the developmental potential is less than or equal to the developmental fate, the cell is exhibiting mosaic behavior. When the developmental potential is greater than the developmental fate, the cell is exhibiting regulative behavior.

Cellular differentiation is also associated with limited cellular proliferation. Indeed, during the development, stem cells are able, under specific condition to be “mobilized” for the self-renewal of the pool of stem cells. Then stem cells proliferate and divide according to the mitosis process, which allows the exact division of a parent cell into two daughter cells comprising the same DNA content, the same morphology and biological and biochemical characteristics.

When stem cells are determined to differentiate, the differentiation process begins by a limited mitotic process, which comprises at least two divisions, but daughter cells progressively acquire, during these limited divisions, the specific feature that they will have at the end of the differentiation process.

So in stem cell niches, which contain the pool of stem cell of an organism or an organ, a balance exists between self-renewal and differentiation.

The process according the invention is implemented preferably in vitro which means that cells are preferably differentiated outside of the organism from which they derive.

By stem cells, it is defined in the invention cells able to differentiate into a diverse range of specialized cell types. These stem cells are defined according to the invention such that they have an intrinsic to differentiate into, from one (unipotent) or two (dipotent) to n (multipotent) differentiated cells, n being more than 2.

The invention concerns pluripotent cells that are the progeny of totipotent cells. In the pluricellular organisms, totipotent cell, which result from the fusion between male and female gamete, is able to differentiate into all the cells that will constitute the organism. The first divisions of this totipotent cell give, by mitosis, some pluripotentent cells. These pluripotent cells have ever acquired a specification, and have lost their ability to give all the differentiated cells.

Therefore stem cells according to the invention concern pluripotent, multipotent, dipotent and unipotent cells. In the invention, the embryonic stem cell (ESC), corresponding to the cell formed by the fusion between male and female gamete can be eventually used.

In one particular embodiment, the embryonic stem cells derived from human, human embryonic stem cells (HESC) are excluded from the use to the implementation of the process of the invention. So, in this particular embodiment, stem cells concern all the animal stem cells provided that said stem cells are not human embryonic stem cells.

According to the invention, terms “stem cells derived from blood or bone marrow or adipose tissue or umbilical cord” mean that stem cells are isolated from the corresponding tissues, i.e. blood or bone marrow or adipose tissue, or umbilical cord, especially from the Wharton's jelly.

In blood, stem cells represent 0.01 and 0.0001% percent of total mononuclear cells [S. S. Khan, M. A. Solomon, J. P. McCoy Jr, Cytometry B Clin. Cytom. 2005, 64, 1]. Classically, mononucleated cells were separated from anucleated cells, i.e. erythrocytes, by a density gradient separation for example. Other methods known in the art are commonly used to separate mononucleated cells. This gradient leads the formation, at the interface of the density gradient, of a ring comprising the mononucleated cells. These “white blood cells” can be cultured in vitro in an appropriate culture medium supplemented with growth factor allowing the proliferation of the endothelial cells [T. Asahara, T. Murohara, A. Sullivan, M. Silver, R. van der Zee, T. Li, B. Witzenbichler, G. Schatteman, J. M. Isner, Science 1997, 275, 964.]. Hematopoietic stem cells, extracted from blood, have the property to bind their support when they are cultured in vitro, and can easily be purified from the other white cells by eliminating unattached cells.

Blood also contains all the stems cells that are able to circulate. For instance, blood also contains Mesenchymal stem cells.

In the invention, blood refers to peripheral blood and placental blood. Commonly, placental blood is obtained from umbilical cord. In the invention placental blood is also called umbilical cord blood. Also, the invention concerns blood contained in tissues and organs.

In bone marrow, three types of stem cells can be found: hematopoietic stem cells, mesenchymal stem cells.

Hematopoietic stem cells are multipotent stem cells able to differentiate into all the circulating white blood cells, such that erythrocyte, macrophages, monocytes . . . .

Mesenchymal stem cells are multipotent cells able to differentiate into all cells of organism i.e. osteoblasts, chondrocytes, myocytes or adipocytes . . . .

In adipose tissue, stem cells, also known as adipose tissue derived stem cells, are able to differentiate into several differentiated cells such as endothelial cells.

In umbilical cord, the Wharton's jelly is a gelatinous substance within the umbilical cord, largely made up of mucopolysaccharides (hyaluronic acid and chondroitin sulfate), that contains, among other cells, adults stem cells, and in particular mesenchymal stem cells.

An “appropriate culture medium”, means a medium comprising nutriments necessary for the survival of cultured cells. This medium has classically pH, glucose concentration, growth factors, and nutrient composition that is specific for in vitro cell survival.

The growth factors used to supplement media are often derived from animal blood, such as calf serum. Moreover, recombinant specific growth factor can be added to specifically initiate a specific cellular process, such as proliferation, differentiation etc. . . .

By “seeded” it is defined in the invention the fact that the cells are deposited on a support and are allowed to attach on said support. It is a common practice in the art, the term seeded concerning in vitro culture cell is commonly used and understood by a skilled person.

In animals, some cells naturally grow without attaching to a surface, such as cells that exist in the bloodstream. Others cells require a surface, such as most cells derived from solid tissues. These adherent cells can be grown on tissue culture plastic, which may be coated with extracellular matrix components to increase its adhesion properties and provide other signals needed for growth.

According to the invention, “specialized differentiated cells” means that these cells have differentiated to a terminal process, and have acquired their complete specialized function. During this process of differentiation, cells begin from stem cells, progressively acquire specific characteristics and functions, and moreover loss progressively their ability to differentiate into different cells. At the terminal steps of the differentiation process, specialized differentiated cells are able to carry out a specific function, (e.g. secretion of hormone, contractibility for muscles . . . ) and remain enable to reverse to the differentiation process. So they are specialized in a function, and differentiated.

According to the invention, “normoxic condition” designates the normal oxygen gas concentration in the environment. Normoxia, which relates to normoxic condition, is the natural composition of air found in earth.

Ambient air is defined in the invention such as the air contained in an environment such as a room, a box, an incubator . . . . The concentration of oxygen in earth is classically around 21%, but varies according to the altitude and the temperature. Then ambient air depends on the location of the experiment.

According to the invention, “hypoxic condition” designates an abnormal oxygen gas concentration found below the normoxic condition. Hypoxia, which relates to the hypoxic condition, corresponds to an oxygen concentration largely reduced compared to the natural concentration. Hypoxia is associated in pathology to asphyxia, and all the pathologies enhanced or induced by a low level of oxygen in the ambient air. The ultimate state of hypoxia is the total absence of O₂ which corresponds to anoxia. The hypoxic conditions according to the invention are different from anoxia, i.e. O₂ is always present even at a very low concentration. For instance in the invention, hypoxia corresponds to low oxygen concentration defined in a range comprised from 0.1% of oxygen to 12% of oxygen.

Classically, a person skilled in cell biology modulates the gas content of its incubator by adding CO₂ gas at known concentration. Indeed, cultured cells are usually grown in an atmosphere comprising from 2 to 15 percent of CO₂. The best CO₂ concentration depends on each cells for providing the best condition for proliferation and/or other cellular process.

Then, with artificial air gas composition, and specific apparatus, a skilled person working on oxygen influence can generate its preferred oxygen-containing culture atmosphere.

According to the invention, the terms “specialized differentiated cells retaining the functional properties of the corresponding specialized differentiated cells respectively obtained through a biological natural process” mean that the specialized differentiated cells obtained by the process of the invention are substantially the same cells as cells taken from an animal.

For example, if the process of the invention allows the differentiation of a stem cell to a specialized differentiated muscle cell, the muscle cell obtained will be able to have a contractility, to produce an extracellular matrix, in the same way as a muscle cell extracted from an animal.

Also, in the invention “the specialized differentiated cells of the first group having cellular functional properties different from the specialized differentiated cells of the second group” means differentiated cells obtained by the differentiation process under nomoxic conditions are functionally different from the cells obtained by the differentiation process under hypoxic conditions. For instance, if a cell differentiates into contractile cells under hypoxic conditions, the same cell under normoxic conditions would differentiate into a cell having a function different from contractibility.

The difference between the two groups of specialized differentiated cells can be easily determined by a skilled person, by optical observation (differences in cell morphology), specific colorations (specific coloration of determined differentiated cells), or by using any methods known in the art that allow, for instance, the identification of membrane markers that are specific of a determined differentiated cell.

The invention also relates to the use of a binary set of two culture media with oxygen specific concentrations culture media, each oxygen specific concentrations culture medium corresponding to a culture medium with a specific oxygen concentration, for the differentiation, preferably in vitro, of stem cells originating from bone marrow or blood or adipose tissue, provided that said stem cells are not human embryonic stem cells and seeded on a support, respectively into:
a first group of specialized differentiated cells by culture of said stem cells on a support in a culture medium under normoxic conditions, and
a second group of specialized differentiated cells by culture of said stem cells on a support in a culture medium of the same nature as the one used for obtaining the first group of specialized differentiated cells, under hypoxic conditions, wherein hypoxic conditions are different from anoxia,
said first and second groups of specialized differentiated cells retaining the functional properties of the corresponding specialized differentiated cells respectively obtained through a biological natural process.
the specialized differentiated cells of the first group having cellular functional properties different from the specialized differentiated cells of the second group.

So the invention relates to the use of a set comprising two culture media with oxygen specific concentrations comprising
two media with nutriments and growth factor necessary for the cell proliferation and differentiation,
two recipients, or surfaces, able to contain each medium, and in which a support is deposited, said support allowing the cell attachment.

These two media differ only by the oxygen concentration in the environment.

The first culture medium with oxygen specific concentrations contain normal oxygen concentration as defined above and the second culture medium with oxygen specific concentrations contain hypoxic oxygen concentrations.

The expression “specific oxygen concentration” means that the oxygen concentration contained in the oxygen specific concentrations culture media comprised in the binary set is known, measured and controlled in order to obtain normoxic conditions or hypoxic conditions.

According to the invention, the first culture medium with oxygen specific concentration is placed under normal oxygen concentrations and provides all the cells required for the cellular differentiation from stem cells to a first group of specialized differentiated cells.

According to the invention, the second culture medium with oxygen specific concentration is placed under hypoxic oxygen concentrations and provides all the cells required for the cellular differentiation from the same stem cell, used in the first oxygen concentration specific medium, to a second group of specialized differentiated cells, said first and second group of specialized differentiated stem cells being such that they are specialized in a particular function different from each other.

Term “support” means any biological or chemical molecules, or polymers, that allow the cell attachment.

Term “surface” defines any recipient or container that can be covered by the above-mentioned support, and liable to contain liquid.

Therefore, when the binary set of the invention is used, stem cells according to the invention, and defined above, are seeded in a support deposited on a surface, said surface being recovered by a nutritive medium comprising nutriment and growth factors, Then, a first part of the stem cells attached in the support deposited on a surface, said surface being recovered by a nutritive medium comprising nutriment and growth factors, is placed in normoxic conditions and allows the differentiation to a first group of specialized differentiated cell, and the remaining part of the stem cells attached in the support deposited on a surface, said surface being recovered by a nutritive medium comprising nutriment and growth factors, is placed in hypoxic conditions and allows the differentiation to a second group of specialized differentiated cell.

As a result of the use of the binary set of oxygen specific according to the invention, only one group of stem cells defined above can provide two distinct specialized differentiated cells that retain the natural properties of the corresponding cells isolated from animal.

In one advantageous embodiment, the invention relates to the uses defined above, wherein normoxic conditions are such that ambient air is constituted by oxygen concentrations comprised from 13% to 21% of molar content per volume (mc/v) of total ambient air gas, preferably from 15 to 20% of molar content per volume (mc/v) of total ambient air gas.

The normoxic conditions correspond to the natural concentration of oxygen contained in earth atmosphere and compatible with life. The Earth's atmosphere is a layer of gases surrounding the planet Earth and retained by the Earth's gravity. It contains roughly (by molar content/volume) 78.08% nitrogen, 20.95% oxygen, 0.93% argon, 0.038% carbon dioxide, trace amounts of other gases, and a variable amount (average around 1%) of water vapor.

The oxygen concentration varying with the pressure and temperature, it is commonly accepted in the art that oxygen concentration in the air is 21+/−1%.

Then natural oxygen concentration for in vitro cell culture is around 20%.

However, the natural oxygen concentration observed in mammal's tissues is lower than the one in ambient air. So, a skilled person in the art commonly modulates the oxygen concentration of a cell culture, by using artificial and known air composition.

So, in cell biology, it is possible to culture cells or cell lines, under a lower oxygen containing atmosphere, for example containing 15% of oxygen. These conditions, although different from the natural oxygen concentrations of the air, are compatible with the normal cell proliferation, without inducing major cellular modification, such as apoptosis or transformation. Then, in cellular biology, the presence of 15%+/−2% of oxygen, depending of the precision of the measurement apparatus, corresponds to normoxic conditions.

In another advantageous embodiment, the invention relates to the uses defined above, wherein hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas.

As defined above, hypoxia, corresponding to low oxygen concentration and also called in the invention hypoxic condition, is defined in a range comprised from 2% of oxygen to 12% of oxygen. On less than 1% of molar content per volume (mc/v) of oxygen, cells are not able to correctly survive and die by necrosis (acute hypoxia). Above 12%, the oxygen concentration is sufficient and the conditions become normoxic.

In another advantageous embodiment, the invention relates to the uses defined above, wherein the support comprises or is constituted by:
Gelatin, fibronectin, collagen, laminin, RGD peptide, or association, or
polyelectrolyte multilayers, preferably polycations and polyanions, preferably alternate,
   said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
   said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
   and preferably chosen among (PAH-PSS)₃, (PAH-PSS)₃-PAH et PEI-(PSS-PAH)₃.
   said support being deposited on a surface.

According to the invention, the stem cells are seeded on a support allowing cell attachment.

This support can be an artificial support that mimic, or reproduce in part, the extracellular matrix on which each cell is attached.

So the support can consist in by recombinant composition of one or more extracellular matrix component.

The extracellular matrix (ECM) is the extracellular part of animal tissue that usually provides structural support to the cells in addition to performing various other important functions. The extracellular matrix is the feature of connective tissue in animals. Components of the ECM are produced intracellularly by resident cells, and secreted into the ECM. Once secreted, they then aggregate with the existing matrix. The ECM consists in of an interlocking mesh of fibrous proteins and glycosaminoglycans (GAG). Fibrous proteins comprised in the ECM are Collagens the most abundant glycoproteins in the ECM, Fibronectins, proteins that connect cells with collagen fibers, elastins, which give the elasticity to tissues, and laminins

Cell adherence on these molecules is well documented in the art: collagen [H. Itoh, Y. Aso, M. Furuse, Y. Noishiki, T. Miyata, Artif. Organs, 25, 213, 2001], la fibronectine [A. Rademacher, M. Paulitschke, R. Meyer, R. Hetzer, Int. J. Artif. Organs, 24, 235, 2001], laminin [A. Sank, K. Rostami, F. Weaver, D. Ertl, A. Yellin, M. Nimni, T. L. Tuan. Am. J. Surg. 164, 199, 1992], la gelatin [J. S. Budd, P. R. Bell, R. F. James. Br. J. Surg. 76, 1259, 1989]. Fibronectin is, to date, the most efficient protein to enhance cell attachment, scattering and retention.

So the support, on which stem cells are seeded, comprises or is constituted by fibronectin, collagen or laminin. Other molecules such as Gelatin or the RGD peptide can also form the support.

RGD peptide corresponds to a tri-peptide of Arginine, Glycine and Aspartic acid.

In the invention, expression “Gelatin, fibronectin, collagen, laminin, RGD peptide, or association” means that the support can comprise or be constituted by one of the above-mentioned molecule, or a combination of at least two of these components. All the compositions, liable to used in the invention, are represented in the following table 1:

| | | | | RGD |
|---|---|---|---|---|
| Gelatin | Fibronectin | Collagen | Laminin | Peptide |
| + | | | | |
| | + | | | |
| | | + | | |
| | | | + | |
| | | | | + |
| + | + | | | |
| + | | + | | |
| + | | | + | |
| + | | | | + |
| | + | + | | |
| | + | | + | |
| | + | | | + |
| | | + | + | |
| | | + | | + |
| | | | + | + |
| + | + | + | | |
| + | + | | + | |
| + | + | | | + |
| + | | + | + | |
| + | | + | | + |
| + | | | + | + |
| | + | + | + | |
| | + | | + | + |
| | | + | + | + |
| + | + | + | + | |
| + | | + | + | + |
| | + | + | + | + |
| + | + | | + | + |
| + | + | + | | + |
| + | + | + | + | + |

Table 1 represents all the combinations of gelatin, fibronectin, collagen, laminin and RGD peptid that can be used as support in the invention.

By <<polyelectrolytes>>, it is defined in the invention polymers wherein monomers have an electrolytic group.

Par <<polyelectrolyte multilayer>>, it is defined according to the invention all the layers obtained by the deposit of polyelectrolytes layers [G. Decher, J. B. Schlenoff, Multilayer thin films: Sequential Assembly of Nanocomposite Materials, Wiley-VCH, Weinheim, 2003].

By <<polycation>>, the invention relates to a polymer with a global positive charge. <<Global positive charge>> means that the total charge is positive, i.e. more than zero, without excluding the fact that monomer can be individually negatively charged.

By <<polyanion>>, the invention relates to polymer with a global negative charge. <<Global negative charge>> means that the total charge is negative, i.e. less than zero, without excluding the fact that monomer can be individually positively charged.

According to another preferred embodiment of the invention, the support can also be constituted by or can comprise polyelectrolytes multilayer chosen among (PAH-PSS)₃, (PAH-PSS)₃-PAH et PEI-(PSS-PAH)₃. [a) H. Kerdjoudj et al. Adv Funct Mater 2007, 17, 2667. b) C. Boura et al. Biomaterials 26, 4568, 2005, c) V Moby et al. Biomacromolecules 2007, 8, 2156]

In another advantageous embodiment, the invention relates to the uses defined above, wherein the layer number of polyelectrolytes layers is from 1 to 100, preferably from 3 to 50, more preferably from 5 to 10, and in particular 7.

Under 7 layers, the thin layer according to the invention remains permeable to small molecules, e.g. Hoechst 33258 (molecular weight 623 Da).

In another advantageous embodiment, the invention relates to the uses defined above, wherein said surface is a natural or artificial surface,
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems, and
said natural surface being chosen among blood vessels, veins, heart, small intestine mucosa, arteries, preferably decellularised umbilical arteries, said vessels, veins, arteries originating from human organs.

In one advantageous embodiment, the invention relates to a natural surface wherein polyelectrolyte multilayers are deposited, said surface being sufficiently rigid to allow cell adhesion and sufficiently flexible to support physiologic deformations. As physiologic deformations, it is meant in the invention, for example, the deformation caused by the arterial pulsatility due to the arterial pressure.

So the surface wherein are deposited polyelectrolyte multilayers are able to resist and to be deformed under physiologic pressure comprised from 10 to 300 mmHg, preferably 50 to 250 mmHg and advantageously 80 to 230 mmHg.

These ranges of pressure have been measured in physiological conditions, in particular in human. For example, in human, if the pressure is upper than 180 mmHg, it is considered as a hypertension condition. Hypotension is defined when pressure is under 50 mmHg.

By <<physiological conditions>>, it is defined in the invention healthy individual blood pressure measured in artery, veins and vessels.

In one preferred embodiment of the invention, the coating of the support deposited on the surface by cells according to the invention is such that it resists to the share stress of blood flow, in particular in vivo.

The <<shear stress of blood flow>>, means, in the invention, the tangential frictional force induced by the blood flow on the combination support and cells covering.

Surfaces used in the invention can be chosen among artificial or natural surfaces.

The “artificial surface” means a surface constituted by materials that do not exit in physiological conditions. For example, an artificial support according to the invention may be glass, plastics, or polymers as defined above. The artificial surface, according to the invention, is compatible with in vitro culture and in vivo cell proliferation. This means that the surface is ascetically prepared in order to prevent bacterial, fungal and viral contaminations.

The surface can have anyone form. In one particular embodiment, the surface used in the invention has a dimension of about at least 20×29 mm, preferably about at least 30×24 mm, and more preferably about at least 300×170 mm, and more preferably about at least 400×200 mm. Surface with a dimension of about 300×170 mm is suitable for the formation of an artificial, i.e. in vitro, functional and transplantable blood vessel. The above-mentioned dimensions are indicated as length×width. In one other particular embodiment, said surface used in the invention is a cell-culture plate or flask, as commonly used in cellular biology by a skilled person. The size of said plate or flask used depends on the desired surface of differentiated cells.

In particular, a plate with dimensions 25×32 mm, preferably 21×29 mm, is used for carrying out the process of the invention.

The surface defined in the invention can also be a natural surface chosen among blood vessels, veins, arteries, preferably decellularised umbilical arteries. According to the invention, placental derma and bladder or any other surface originating from organs can also be used in the invention.

Natural surfaces used in the invention originate from animal or human organs or tissues.

It is also important to note that the surface defined in the invention, wherein is deposited the support defined above can be separated by a removable material sufficiently rigid to allow the separation of cells on support from surface, and sufficiently flexible to be wrapped around a stick, without breaking the support containing cells.

In another advantageous embodiment, the invention relates to the uses defined above, wherein said stem cells are chosen among mesenchymal stem cells (MSC) and hematopoietic stem cells (HSC).

According to the invention, the stem cells used in the invention can be chosen among hematopoietic stem cells or mesenchymal stem cells. Preferably, the stem cells used in the invention are hematopoietic stem cells.

HSC are found in the adult bone marrow, including bone marrow of femurs, hip, ribs, sternum, and other bones. HSC can be obtained directly by removal from the hip using a needle and syringe, or from the blood following pre-treatment with cytokines, such as G-CSF (granulocyte colony-stimulating factors), that induce cells to be released from the bone marrow compartment. Other sources for clinical and scientific use include umbilical cord blood, placenta, and mobilized peripheral blood. For experimental purposes, fetal liver and fetal spleen of animals are also useful sources of HSC.

It is now well documented that HSC derive from hemangioblast multipotent cells, which are also the precursor of endothelial cells. It has been shown that these pre-endothelial/pre-hematopoietic cells in the embryo arise out of a phenotype CD34 population. It was then found that hemangioblasts are also present in the tissue of fully developed individuals, such as in newborn infants and adults.

There is now emerging evidence of hemangioblasts that continue to exist in the adult as circulating stem cells in the peripheral blood can give rise to both endothelial cells and hematopoietic cells. These cells are thought to express both CD34 and CD133. These cells are likely derived from the bone marrow, and may even be derived from hematopoietic stem cells.

In another advantageous embodiment, the invention relates to the uses defined above, wherein the first and the second groups of specialized differentiated cells consist of cells chosen among endothelial cells and smooth muscle cells.

According to the invention, smooth muscle cells are defined such that they participate in the formation of a smooth muscle, which is a type of non-striated muscle, found for example, in arteries and veins. The cells are arranged in sheets or bundles and connected by gap junctions. In order to contract, the cells contain actin filaments and a contractous protein called myosin. Whereas the filaments are essentially the same in smooth muscle as they are in skeletal and cardiac muscle, the way they are arranged is different.

Smooth muscle cells may secrete their own complex extracellular matrix containing collagen (predominantly types I and III), elastin, glycoproteins, and proteoglycans [Rzucidlo, E. M., Martin, K. A. & Powell, R. J. Regulation of vascular smooth muscle cell differentiation. J Vasc Surg. 45, 25-32 (2007).]. These fibers with their extracellular matrices contribute to the viscoelasticity of these tissues. Smooth muscle also has specific elastin and collagen receptors which interact with these proteins.

The contractile function of vascular smooth muscle is critical for regulating the lumenal diameter of the small arteries-arterioles called resistance vessels. The resistance arteries contribute significantly to the setting of the level of blood pressure. Smooth muscle contracts slowly and may maintain the contraction. From the biochemical content of cells, smooth muscle cells express specific proteins, involved in the contraction, such as smooth muscle actin, smooth muscle myosin and desmin.

So in the invention, the essential functional properties of smooth muscle cells are the secretion of extracellular matrix component mentioned above, and the contractibility potential. These properties are the properties found in the biological natural process of smooth muscle cells.

According to the invention, endothelial cells form the thin layer of cells (endothelium) that line the interior surface of blood vessels, forming an interface between circulating blood in the lumen and the rest of the vessel wall. The endothelium is composed of a single layer of endothelial cells.

Endothelial cells play an essential role in the vascular development and in the preservation of the vessel functions. Once vessels were formed, endothelial cells control the vascular tonus, by leading a vasodilatation or a vasoconstriction according to the conditions, so maintaining the degree of mechanical constraint of the wall at constant levels. They also can participate to the in vivo neo-vascularization.

In another advantageous embodiment, the invention relates to the uses defined above, wherein said first group of specialized differentiated cells consists of endothelial cells and said second group of specialized differentiated cells consists of smooth muscle cells.

So according to the invention, the stem cells cultured according to the process of the invention differentiate into:
Endothelial cells, when they are grown in normoxic conditions as defined above, or
Smooth muscle cells, when they are grown in hypoxic conditions as defined above.

An advantageous embodiment of the invention relates to the use of specific oxygen concentrations for implementing an in vitro process of differentiation of
either mesenchymal stem cells
or hematopoietic stem cells
   seeded on a support, said support deposited on a surface comprising or being constituted by:
Gelatin, fibronectin, collagen, laminin, RGD peptide, or association, or
polyelectrolyte multilayers, preferably polycations and polyanions, preferably alternate,
   said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
   said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
   and preferably chosen among (PAH-PSS)₃, (PAH-PSS)₃-PAH et PEI-(PSS-PAH)₃.
   said support being deposited on a surface, said surface being a natural or artificial surface, wherein:
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems,
said natural surface being chosen among blood vessels, veins, heart, small intestinal submucosa, arteries, preferably decellularised umbilical arteries, said vessels, veins, arteries originating from human organs.
   in an appropriate culture medium,
   wherein said differentiation leads to:
a first group of specialized differentiated cells under normoxic conditions, and in an appropriate culture medium, wherein said first group of specialized differentiated cells consists of endothelial cells and
a second group of specialized differentiated cells under hypoxic conditions, wherein hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas, in a culture medium of the same nature as the one used for obtaining the first group of specialized differentiated cells, said second group of specialized differentiated cells consists of smooth muscle cells.

Another advantageous embodiment of the invention relates to the use of a binary set of two culture media with oxygen specific concentrations culture media, each oxygen specific concentrations culture medium corresponding to a culture medium with specific oxygen concentrations, for the differentiation of:
either mesenchymal stem cells
or hematopoietic stem cells
   seeded on a support, said support deposited on a surface comprising or being constituted by:
Gelatin, fibronectin, collagen, laminin, RGD peptide, or association, or
polyelectrolyte multilayers, preferably polycations and polyanions, preferably alternate,
   said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
   said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
   and preferably chosen among (PAH-PSS)₃, (PAH-PSS)₃-PAH et PEI-(PSS-PAH)₃.
   said support being deposited on a surface, said surface being a natural or artificial surface, wherein:
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems,
said natural surface being chosen among blood vessels, veins, heart, small intestinal submucosa, arteries, preferably decellularised umbilical arteries, said vessels, veins, arteries originating from human organs.
   in an appropriate culture medium,
   wherein said differentiation leads to:
a first group of specialized differentiated cells under normoxic conditions, and in an appropriate culture medium, wherein said first group of specialized differentiated cells consists of endothelial cells, and
a second group of specialized differentiated cells under hypoxic conditions, wherein hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas, in a culture medium of the same nature as the one used for obtaining the first group of specialized differentiated cells, said second group of specialized differentiated cells consists of smooth muscle cells.

The invention also relates to a culture medium with oxygen specific concentrations culture medium comprising:
an appropriate culture medium, and
oxygen atmosphere concentrations in said culture medium comprised from 2% to 12% of molar content per volume (mc/v) of total air, preferably from 3 to 8% of molar content per volume (mc/v) of total air, and more preferably from 4 to 6% of molar content per volume (mc/v) of total air.

The invention then relates to culture medium with oxygen specific concentrations comprising nutriments essential for cell survival, such as sugar, amino acid, vitamins . . . . This medium is complemented with growth factor originating from animal serum, or recombinant growth factor. As culture medium, it is possible to use, without limiting to, the following available medium: α-MEM, DMEM, RPMI 1640, Iscove's medium, Mac Coy medium, EBM-2 medium, etc. . . .

Moreover this medium is conditioned such that the oxygen concentration that it comprises corresponds to hypoxic condition.

In the invention, the oxygen concentration of the oxygen specific concentrations culture medium can be controlled by any chemical or biological compound or molecule liable to diffuse in the culture medium an oxygen concentration comprised from 2% to 12% of oxygen. In one particular embodiment, the oxygen specific concentration culture medium according to the invention can consist of a culture medium described above placed in a hermetically closed space wherein oxygen concentration is controlled.

In one advantageous embodiment, the invention relates to a culture medium with oxygen specific concentrations defined above, in association with a support deposited on a surface.

The invention relates also to a culture medium with oxygen specific concentrations comprising:
an appropriate culture medium,
oxygen at concentrations in said culture medium comprised from 13% to around 21% of molar content per volume (mc/v) of total ambient air gas, preferably from 15 to 21% of molar content per volume (mc/v) of total ambient air gas.
in association with a support deposited on a surface.

The invention also relates to a binary set of two culture media with oxygen specific concentration, each oxygen specific concentration culture medium corresponding to an appropriate culture medium and specific oxygen concentrations, comprising:
an appropriate culture medium with oxygen at concentrations in said culture medium comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas, in association with a support deposited on a surface, and
an appropriate culture medium with oxygen at concentrations in said culture medium comprised from 13% to 10% of molar content per volume (mc/v) of total ambient air gas, in association with a support deposited on a surface.

According to the invention, the binary set of two culture media with oxygen specific concentration comprises, or is constituted by, a first appropriate culture medium comprising nutriments, growth factors . . . for cell survival, placed under hypoxic condition, and a second appropriate culture medium of the same nature as the first appropriate culture medium.

“A second appropriate culture medium of the same nature than the first appropriate culture medium” means in the invention that the first and the second appropriate culture medium have exactly the same composition in term of constituents, i.e. the two appropriate medium comprises the same nutriments, growth factors . . . .

In one advantageous embodiment, the invention relates to a culture medium with oxygen specific concentration defined above, or a binary set of two culture media with oxygen specific concentrations defined above, wherein said support deposited on a surface comprises or is constituted by:
Gelatin, fibronectin, collagen, laminin, RGD peptide, or association, or
polyelectrolytes multilayers, preferably polycations and polyanions, preferably alternate,
   said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such that polylysine and polysaccharides negatively charged such that chitosane, and
   said polyanions being chosen among the group comprising: polyacrylic acid (PAA), polymetacrylic acid (PMA), polystyrene sulfonic acid (PSS or SPS), negatively charged polypeptides such that polyglutamic acid and polyaspartic acid and polysaccharides negatively charged such that hyaluronan and alginate,
   and preferably chosen among (PAH-PSS)₃, (PAH-PSS)₃-PAH et PEI-(PSS-PAH)₃.

In one advantageous embodiment, the invention relates to a culture medium with oxygen specific concentrations or binary set of two culture media with oxygen specific concentration defined above, wherein said surface is a natural or artificial surface
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems or cultured system,
said natural surface being chosen among blood vessels, veins, heart, small intestine mucosa, arteries, preferably decellularised umbilical artery, said vessels, veins, arteries derived from human organs.

The invention relates to a process of differentiation of stem cells derived from bone marrow or blood, or adipose tissue, comprising:
contacting stem cells originating from bone marrow or blood, or adipose tissue, or umbilical cord with a support deposited on a surface in an appropriate culture medium, to obtain seeded stem cells on a support,
varying oxygen concentrations in said appropriate culture medium containing said seeded stem cells on the support, to provide normoxic or hypoxic conditions,
leaving the achievement of the in vitro differentiation of said seeded stem cells on the support,
   either into a first group of specialized differentiated cells by culture of said seeded stem cells on a support under normoxic conditions,
   or into a second group of specialized differentiated cells by culture of said seeded stem cells on a support, in a culture medium of the same nature as the one used for obtaining the first group of specialized differentiated cells, under hypoxic conditions,
said first and second groups of specialized differentiated cells retaining the functional properties of the corresponding specialized differentiated cells respectively obtained through a biological natural process.

The invention relates to a process of in vitro differentiation of stem cells, derived from bone marrow or blood, or adipose tissue, or umbilical cord, provided that said stem cells are not human embryonic stem cells, and are preferably chosen among mesenchymatous stem cells (MSC) and hematopoietic stem cells (HSC) comprising:
contacting stem cells originating from bone marrow or blood, or adipose tissue, provided that said stem cells are not human embryonic stem cells, with a support deposited on a surface in an appropriate culture medium, to obtain seeded stem cells on a support,
varying oxygen concentrations in said appropriate culture medium containing said seeded stem cells on the support, to provide normoxic or hypoxic conditions, said hypoxic conditions being different from anoxia
leaving the achievement of the in vitro differentiation of said seeded stem cells on the support,
   either into a first group of specialized differentiated cells by culture of said seeded stem cells on a support under normoxic conditions,
   or into a second group of specialized differentiated cells by culture of said seeded stem cells on a support, in a culture medium of the same nature as the one used for obtaining the first group of specialized differentiated cells, under hypoxic conditions,
said first and second groups of specialized differentiated cells retaining the functional properties of the corresponding specialized differentiated cells respectively obtained through a biological natural process.
the specialized differentiated cells of the first group having cellular functional properties different from the specialized differentiated cells of the second group.

Stem cells originating from the selected organ or body fluid defined above are seeded in two different surfaces covered by a support defined above and coated by the appropriate culture medium. The attached stem cells were separated from the unattached cells and left in a culture incubator for 1 to 10 days, preferably 4 days, at 37° C.

Further, oxygen concentration of one surface coated by support covered by appropriate culture medium wherein stem cells are seeded is placed in an hypoxic atmosphere, whereas the other surface coated by support covered by appropriate culture medium wherein stem cells are seeded is placed under normoxic atmosphere.

Then the cells are left in the corresponding atmosphere until the complete achievement of the respective cellular differentiation process. According to the invention, the complete differentiation process is achieved after 10 to 20 days, preferably 11 to 18 days, more preferably after 14 days.

After this time, cells grown under normoxic conditions are differentiated in a first group of specialized differentiated cells, and the cells grown under hypoxic conditions are differentiated in a second group of specialized differentiated cell.

Classical phetontyping technics can be used to characterize the nature of specialized differentiated cells obtained according to the process of the invention, such as immunophenotyping, PCR, immunohistochemistry . . . .

The inventions also relates to a process of functional blood vessel formation using a binary set of two oxygen specific concentration culture media, each oxygen specific concentration culture medium corresponding to an appropriate culture medium with specific oxygen concentrations,
said process comprising the following steps:
contacting said stem cells derived from bone marrow or blood, or adipose tissue, with a support deposited on a surface in an appropriate culture medium, to obtain seeded stem cells on a support,
varying oxygen concentrations in said appropriate culture medium containing seeded stem cells on a support, to provide normoxic or hypoxic conditions, said hypoxic conditions being different from anoxia
leaving the achievement of the in vitro differentiation of said seeded stem cells on a support, respectively into:
   a first group of specialized differentiated cells by culture of said seeded stem cells on a support in a culture medium under normoxic conditions, and
   a second group of specialized differentiated cells by culture of said seeded stem cells on a support in a culture medium of the same nature as the one used for obtaining the first group of specialized differentiated cells, under hypoxic conditions,
collecting respectively the first and the second group of specialized differentiated cells, and
building-up a vessel constituted by a second group of specialized differentiated cells layers outside, and a first group of specialized differentiated cells monolayer inside, and limiting the lumen, and hence allowing the formation of a functional blood vessel.

The inventions also relates to a process of in vitro functional blood vessel formation using a binary set of two culture media with oxygen specific concentration, each culture medium with oxygen specific concentration corresponding to an appropriate culture medium with specific oxygen concentrations,
said process comprising the following steps:
contacting said stem cells derived from bone marrow or blood, or adipose tissue, provided that said stem cells are not human embryonic stem cells, with a support deposited on a surface in an appropriate culture medium, to obtain seeded stem cells on a support,
varying oxygen concentrations in said appropriate culture medium containing seeded stem cells on a support, to provide normoxic or hypoxic conditions, said hypoxic conditions being different from anoxia
leaving the achievement of the in vitro differentiation of said seeded stem cells on a support, respectively into:
   a first group of specialized differentiated cells by culture of said seeded stem cells on a support in a culture medium under normoxic conditions, and
   a second group of specialized differentiated cells by culture of said seeded stem cells on a support in a culture medium of the same nature as the one used for obtaining the first group of specialized differentiated cells, under hypoxic conditions,
collecting respectively the first and the second group of specialized differentiated cells, and
building-up a vessel constituted by a second group of specialized differentiated cells layers outside, and a first group of specialized differentiated cells monolayer inside, and limiting the lumen, and hence allowing the formation of a functional blood vessel.

According to the invention, the process described above allows the formation, preferably in vitro, of a functional, transplantable and immunologically compatible blood vessel.

The process described above allow the differentiation, according to either hypoxic or normoxic conditions, to two different specialized differentiated cells.

The first group of specialized differentiated cells is grown, under normoxic condition, in order to completely cover the surface recovered by the support.

The second group of specialized differentiated cells is grown, under hypoxic condition, in order to completely cover the surface recovered by the support. The surface can have anyone form. In one particular embodiment, the surface used in the invention has a dimension of about at least 20×29 mm, preferably about at least 30×24 mm, and more preferably about at least 300×170 mm, and more preferably about at least 400×200 mm. Surface with a dimension of about 300×170 mm is suitable for the formation of an artificial, i.e. in vitro, functional and transplantable blood vessel. The above-mentioned dimensions are indicated as length×width.

Preferably, the support wherein are seeded stem cells grown under hypoxic condition is easily removable from the surface. This step corresponds to the recovery of the second group of specialized differentiated cells.

The recovery of the second group of cells is made such that it does not destroy the layer form by the cells.

Then the recovered layer is rolled up around itself by using a stick. The stick used previously is such that it does not allow the cell adhesion, and is for example a Teflon stick. The stick allows to maintain the lumen of the formed tube.

Then the rolled layer is leaved from about 2 to about 45 days, and placed in a bioreactor to be submitted to mechanical stains.

Then, the first group of specialized differentiated cells according to the invention is recovered by classical techniques used by skilled persons. For example, cells can be treated with trypsin, EDTA, or placed on ice, or scratched. The above example allows the recovery of said first group of specialized differentiated cells.

Then, the first group of specialized differentiated cells is placed in the lumen of the tube formed by the rolling up of the layer of the second group of specialized differentiated cells.

So specialized differentiated cells the first of the group adhere the inner face of the tube, and a blood vessel is now formed.

In one advantageous embodiment, the invention relates to processes defines above, wherein:
said normoxic conditions are such that ambient air is constituted by oxygen concentrations comprised from 13% to 21% of molar content per volume (mc/v) of total ambient air gas, preferably from 15 to 21% of molar content per volume (mc/v) of total ambient air gas, and
said hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas.

In another advantageous embodiment, the invention relates to processes defined above, wherein said support comprises or is constituted by:
Gelatin, fibronectin, collagen, laminin, RGD peptide, or association, or
polyelectrolytes multilayers, preferably polycations and polyanions, preferably alternate,
   said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such that polylysine and polysaccharides negatively charged such that chitosane, and
   said polyanions being chosen among the group comprising: polyacrylic acid (PAA), polymetacrylic acid (PMA), polystyrene sulfonic acid (PSS or SPS), negatively charged polypeptides such that polyglutamic acid and polyaspartic acid and polysaccharides negatively charged such that hyaluronan and alginate,
   and preferably chosen among (PAH-PSS)₃, (PAH-PSS)₃-PAH et PEI-(PSS-PAH)₃.
   said support being deposited on a surface.

In another advantageous embodiment, the invention relates to processes defined above, wherein said surface is a natural or artificial surface,
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, polytetrafluoroethylene (PTFEe), and any material used for prothesis and/or implanted systems,
said natural surface being chosen among blood vessels, veins, heart, small intestine mucosa, arteries, preferably decellularised umbilical arteries, said vessels, veins, arteries originating from human organs.

In another advantageous embodiment, the invention relates to processes defined above, wherein said stem cells are chosen among mesenchymatous stem cells (MSC) and hematopoietic stem cells (HSC).

In another advantageous embodiment, the invention relates to processes defined above, wherein the first and the second group of specialized differentiated cells consist of cells chosen among endothelial cells and smooth muscle cells.

In another advantageous embodiment, the invention relates to processes defined above, wherein said first group of specialized differentiated cells consists of endothelial cells and said second group of specialized differentiated cells consists of smooth muscle cells.

The invention also relates to a process of transdifferentiation of stem cells derived from bone marrow or blood, or adipose tissue, comprising:
contacting said stem cells derived from bone marrow or blood, or adipose tissue, or umbilical cord, with a support deposited on a surface in an appropriate culture medium, to obtain seeded stem cells on a support,
varying oxygen concentrations in said appropriate culture medium containing seeded stem cells on a support, to provide normoxic or hypoxic conditions,
leaving said seeded stem cells on a support starting the in vitro differentiation, respectively into:
   a first group of specialized differentiated cells by culture of said seeded stem cells on a support in a culture medium under normoxic conditions, and
   a second group of specialized differentiated cells by culture of said seeded stem cells on a support in a culture medium of the same nature as the one used for obtaining the first group of specialized differentiated cells, under hypoxic conditions, said hypoxic conditions being different from anoxia
changing oxygen concentrations in the respective culture medium of the first and the second group above defined, such that
   cells that have started the differentiation process into a first group of specialized differentiated cells are placed under hypoxic conditions, and
   cells that have started the differentiation process into a second group of specialized differentiated cells are placed under normoxic conditions,
leaving the achievement of the in vitro differentiation of said seeded stem cells that have started a differentiation process under normoxia or hypoxia, and have been placed under hypoxia or normoxia respectively, to obtain
   a third group of specialized differentiated cells by culture of said seeded stem cells of the second group on a support in a culture medium under normoxic conditions, and
   a fourth group of specialized differentiated cells by culture of said seeded stem cells of the first group on a support in a culture medium of the same nature as the one used for obtaining the first group of specialized differentiated cells, under hypoxic conditions,
said first, second, third and fourth groups of specialized differentiated cells retaining the functional properties of the corresponding specialized differentiated cells respectively obtained through a biological natural process.

The invention also relates to a process of transdifferentiation, preferably in vitro, of stem cells derived from bone marrow or blood, or adipose tissue, or umbilical cord, provided that said stem cells are not human embryonic stem cells, comprising:
contacting said stem cells derived from bone marrow or blood, or adipose tissue, provided that said stem cells are not human embryonic stem cells, with a support deposited on a surface in an appropriate culture medium, to obtain seeded stem cells on a support,
varying oxygen concentrations in said appropriate culture medium containing seeded stem cells on a support, to provide normoxic or hypoxic conditions,
leaving said seeded stem cells on a support starting the in vitro differentiation, respectively into:
   a first group of specialized differentiated cells by culture of said seeded stem cells on a support in a culture medium under normoxic conditions, and
   a second group of specialized differentiated cells by culture of said seeded stem cells on a support in a culture medium of the same nature as the one used for obtaining the first group of specialized differentiated cells, under hypoxic conditions
changing oxygen concentrations in the respective culture medium of the first and the second group above defined, such that
   cells that have started the differentiation process into a first group of specialized differentiated cells are placed under hypoxic conditions, and
   cells that have started the differentiation process into a second group of specialized differentiated cells are placed under normoxic conditions,
leaving the achievement of the in vitro differentiation of said seeded stem cells that have started a differentiation process under normoxia or hypoxia, and have been placed under hypoxia or normoxia respectively, to obtain
   a third group of specialized differentiated cells by culture of said seeded stem cells of the second group on a support in a culture medium under normoxic conditions, and
   a fourth group of specialized differentiated cells by culture of said seeded stem cells of the first group on a support in a culture medium of the same nature as the one used for obtaining the first group of specialized differentiated cells, under hypoxic conditions,
said first, second, third and fourth groups of specialized differentiated cells retaining the functional properties of the corresponding specialized differentiated cells respectively obtained through a biological natural process.

Expression “transdifferentiation” means that cells are able to reverse the differentiation process they have started. In particular, transdifferentiation in the invention means that cells retain the ability to reverse the differentiation process and are able to differentiate into another cellular subtype, different from the one from which they have started.

For instance, the stem cells placed under hypoxic conditions start a differenciation process to give a first group of specialized differentiated cells. But before the end of the differentiation process, the oxygen concentrations are changed, and cells are placed under normoxic conditions. Then, stem cells will differentiate into a fourth group of specialized differentiated cells, as if they had directly started the differentiation process under normoxic conditions. So, the fourth group of specialized differentiated cells is substantially the same as the second group of specialized differentiated.

For instance, the stem cells placed under normoxic conditions start a differentiation process to give a second group of specialized differentiated cells. But before the end of the differentiation process, the oxygen concentrations are changed, and cells are placed under hypoxic conditions. Then, stem cells will differentiate into a third group of specialized differentiated cells, as if they had directly started the differentiation process under hypoxic conditions. So, the third group of specialized differentiated cells is substantially the same as the first group of specialized differentiated.

The invention relates to a process of differentiation, preferably in vitro, of hematopoietic stem cells derived from bone marrow or blood, into smooth muscle cells comprising:
contacting hematopoietic stem cells originating from bone marrow or blood, with a support deposited on a surface in an appropriate culture medium, to obtain seeded stem cells on a support,
varying oxygen concentrations in said appropriate culture medium containing said seeded stem cells on the support, to provide hypoxic conditions,
leaving the achievement of the in vitro differentiation of said seeded hematopoietic stem cells on the support into smooth muscle cells,
said smooth muscle cells retaining the functional properties of the corresponding smooth muscle cells obtained through a biological natural differentiation process.

The invention described above is explained and illustrated, but not limited to, by the following examples and the following figures.

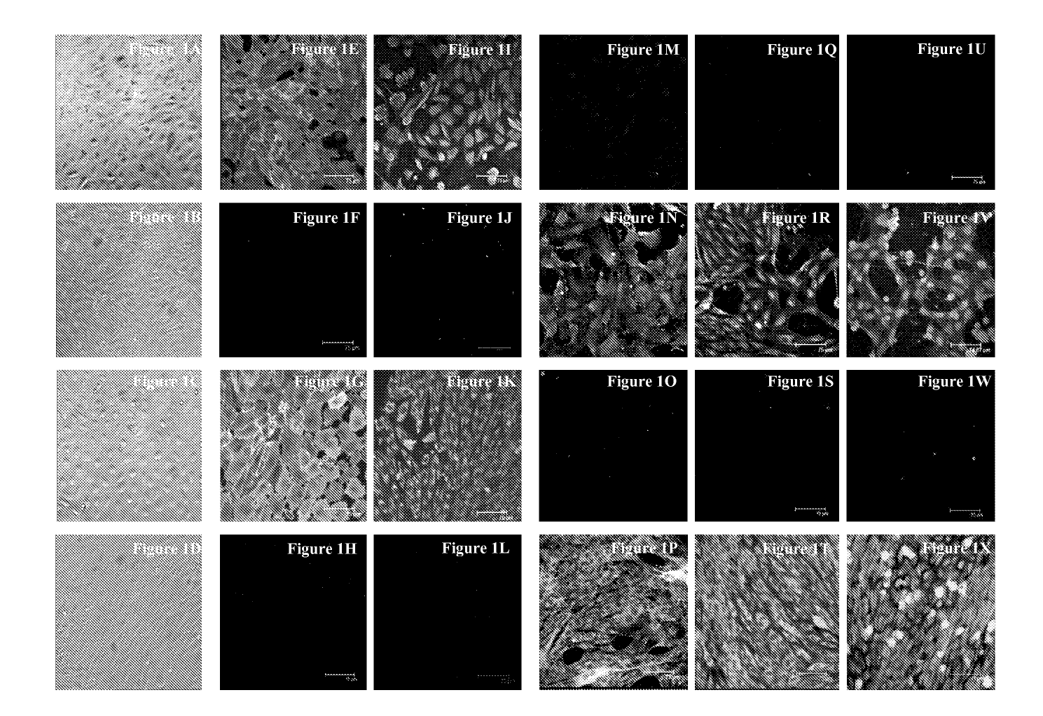

FIGS. 1A-X represent morphological observation by optical phase contrast microscopy (Objective×20), or immunofluorescent phenotype characterization by confocal microscopy observation (Objective×40) of cells seeded on type I collagen and PME until confluence under normoxia environment and under hypoxic environment. Results show the positive expression of specific SMC contractile markers (α-actin; SM-MHC; calponin) or specific endothelial cells markers (CD31; vWF). NA=0.8, scale bars 75 μm.

More precisely:

FIG. 1A represents optical phase observation of cells seeded on type I collagen and placed under normoxic conditions.

FIG. 1B represents optical phase observation of cells seeded on type I collagen and placed under hypoxic conditions.

FIG. 1C represents optical phase observation of cells seeded on PEM and placed under normoxic conditions.

FIG. 1D represents optical phase observation of cells seeded on PEM and placed under hypoxic conditions.

FIG. 1E represents fluorescent immunostaining of cells seeded on type I collagen and placed under normoxic conditions with an anti-CD31 antibody, and observation by confocal microscopy.

FIG. 1F represents fluorescent immunostaining of cells seeded on type I collagen and placed under hypoxic conditions with an anti-CD31 antibody, and observation by confocal microscopy.

FIG. 1G represents fluorescent immunostaining of cells seeded on PEM and placed under normoxic conditions with an anti-CD31 antibody, and observation by confocal microscopy.

FIG. 1H represents fluorescent immunostaining of cells seeded on PEM and placed under normoxic conditions with an anti-CD31 antibody, and observation by confocal microscopy.

FIG. 1I represents fluorescent immunostaining of cells seeded on type I collagen and placed under normoxic conditions with an anti-vWF antibody, and observation by confocal microscopy.

FIG. 1J represents fluorescent immunostaining of cells seeded on type I collagen and placed under hypoxic conditions with an anti-vWF antibody, and observation by confocal microscopy.

FIG. 1K represents fluorescent immunostaining of cells seeded on PEM and placed under normoxic conditions with an anti-vWF antibody, and observation by confocal microscopy.

FIG. 1L represents fluorescent immunostaining of cells seeded on PEM and placed under normoxic conditions with an anti-vWF antibody, and observation by confocal microscopy.

FIG. 1M represents fluorescent immunostaining of cells seeded on type I collagen and placed under normoxic conditions with an anti-α actin antibody, and observation by confocal microscopy.

FIG. 1N represents fluorescent immunostaining of cells seeded on type I collagen and placed under hypoxic conditions with an anti-α actin antibody, and observation by confocal microscopy.

FIG. 1O represents fluorescent immunostaining of cells seeded on PEM and placed under normoxic conditions with an anti-α actin antibody, and observation by confocal microscopy.

FIG. 1P represents fluorescent immunostaining of cells seeded on PEM and placed under normoxic conditions with an anti-α actin antibody, and observation by confocal microscopy.

FIG. 1Q represents fluorescent immunostaining of cells seeded on type I collagen and placed under normoxic conditions with an anti-Smooth Muscle-Myosin Heavy Chain (SM-MHC) antibody, and observation by confocal microscopy.

FIG. 1R represents fluorescent immunostaining of cells seeded on type I collagen and placed under hypoxic conditions with an anti Smooth Muscle-Myosin Heavy Chain (SM-MHC) antibody, and observation by confocal microscopy.

FIG. 1S represents fluorescent immunostaining of cells seeded on PEM and placed under normoxic conditions with an anti-Smooth Muscle-Myosin Heavy Chain (SM-MHC) antibody, and observation by confocal microscopy.

FIG. 1T represents fluorescent immunostaining of cells seeded on PEM and placed under normoxic conditions with an anti-Smooth Muscle-Myosin Heavy Chain (SM-MHC) antibody, and observation by confocal microscopy.

FIG. 1U represents fluorescent immunostaining of cells seeded on type I collagen and placed under normoxic conditions with an anti-Calponin antibody, and observation by confocal microscopy.

FIG. 1V represents fluorescent immunostaining of cells seeded on type I collagen and placed under hypoxic conditions with an anti-Calponin antibody, and observation by confocal microscopy.

FIG. 1W represents fluorescent immunostaining of cells seeded on PEM and placed under normoxic conditions with an anti-Calponin antibody, and observation by confocal microscopy.

FIG. 1X represents fluorescent immunostaining of cells seeded on PEM and placed under normoxic conditions with an anti-Calponin antibody, and observation by confocal microscopy.

FIGS. 2A-D represent confocal microscopy observations of Extracellular matrix (ECM) proteins and cytoskeleton secretion of smooth muscle cells differentiated on type I collagen or on PEM. Objective×40, NA=0.8, scale bars 75 μm.

More precisely:

FIG. 2A represents fluorescent immunostaining of smooth muscle cells with an anti-laminin antibody, and observation by confocal microscopy, seeded on type I collagen, and differentiated under hypoxic conditions.

FIG. 2B represents fluorescent immunostaining of smooth muscle cells with an anti-laminin antibody, and observation by confocal microscopy, seeded on PEM, and differentiated under hypoxic conditions.

FIG. 2C represents fluorescent immunostaining of smooth muscle cells with an anti-type IV collagen antibody, and observation by confocal microscopy, seeded on type I collagen, and differentiated under hypoxic conditions.

FIG. 2D represents fluorescent immunostaining of smooth muscle cells with an anti-type IV collagen, and observation by confocal microscopy, seeded on PEM, and differentiated under hypoxic conditions.

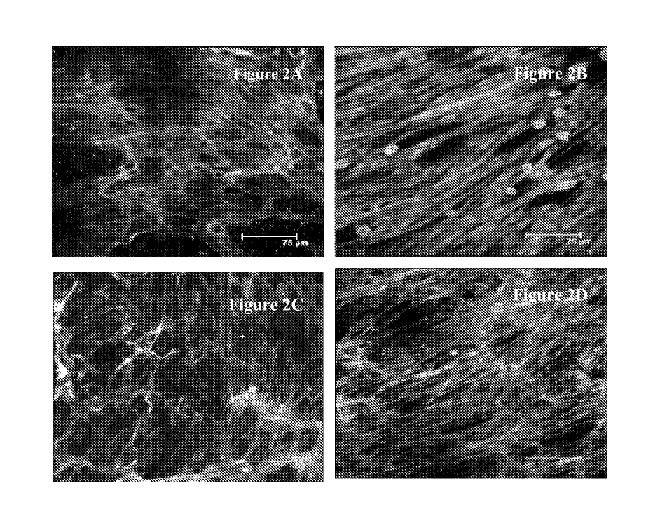

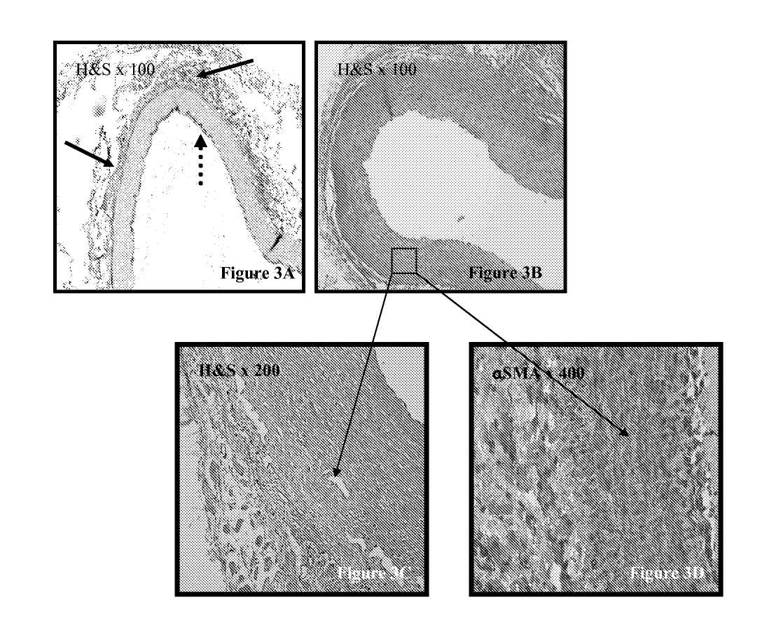

FIGS. 3A-C represent histological cross sections of rabbit carotid arteries treated with PEM.

Magnification is indicated on figures.

FIG. 3A represents histological cross sections, colored with H&S (Haematoxylin, Eosin, Safran), of rabbit carotid arteries treated with PEM at 1 week post-surgery. Blacks arrows indicate the presence of inflammatory cells and dotted arrow indicate the PEM deposition into the luminal surface of artery.

FIG. 3B represents histological cross sections, colored with H&S (Haematoxylin, Eosin, Safran), of rabbit carotid arteries treated with PEM at 12 weeks post-surgery. The insert (black square) represents an enlargement of the section

FIG. 3C represents an enlargement (×2) of a region of rabbit carotid arteries treated with PEM at 12 weeks post-surgery and highlighted the vasa vasorum formation.

FIG. 3D represents the immunohistochemical study of the enlarged region, performed on deparaffinized sections after epitope restoration, and labelled with anti-Smooth Muscle a Actin antibody.

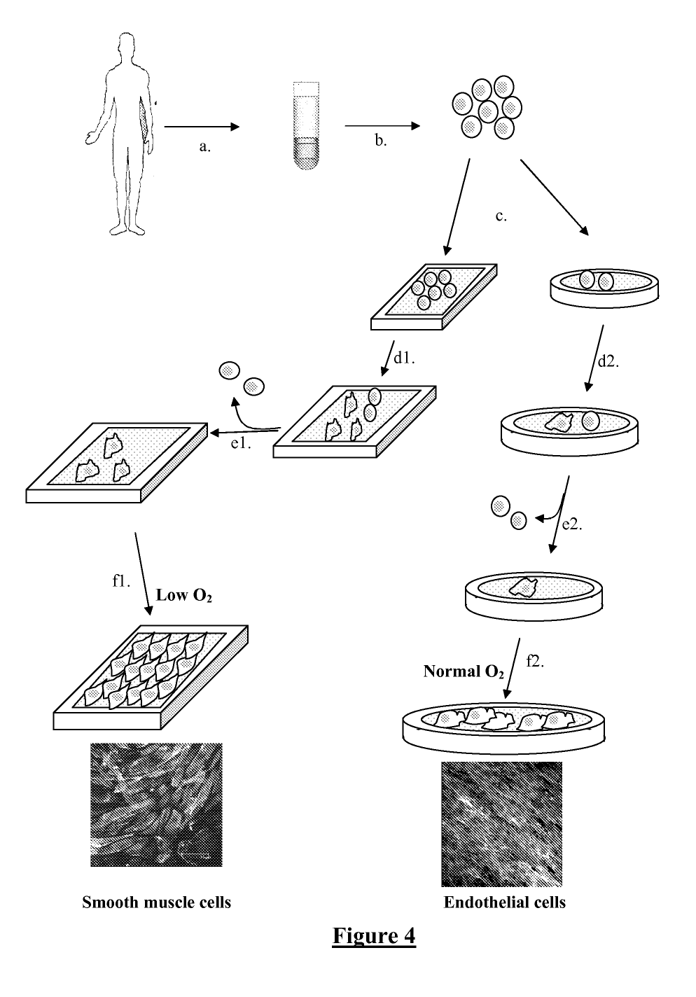

FIG. 4 represents the steps for preparing smooth muscles cells and endothelial cells from blood sample. Doted area [symbol] represents surface covered by the support of the invention.

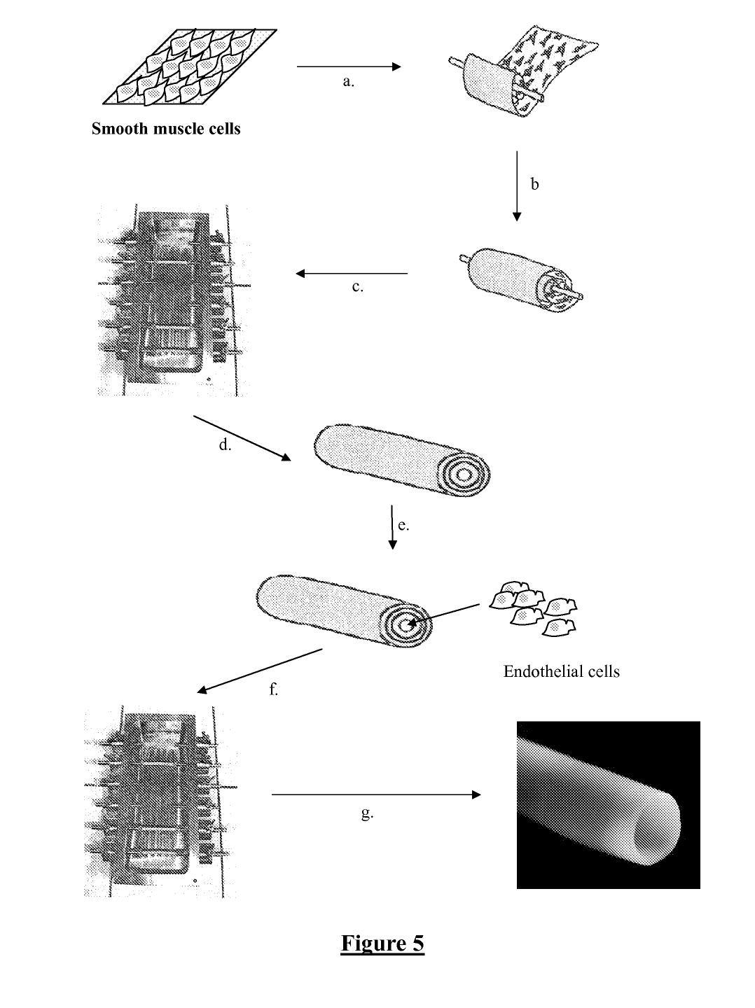

FIG. 5 represents the physical modifications applied to the surface covered by the support, for the formation of an artificial blood vessel.

FIGS. 6A-F represent the phenotype stability under hypoxia analysed by confocal microscopy after immunostaining with contractile markers α-Smooth Muscle Actin (α-SMA), Smooth Muscle Myosin Heavy Chain (SM-MHC) and Calponin antibodies on both coated surfaces (type I collagen and Polyelectrolyte Multilayer films (PEMs)). Objective×40, NA=0.8, scale bars 75 μm.

FIG. 6A represents fluorescent immunostaining of smooth muscle cells with an anti-α-Smooth Muscle Actin antibody, and observation by confocal microscopy, seeded on type I collagen.

FIG. 6B represents fluorescent immunostaining of smooth muscle cells with an anti-Smooth Muscle Myosin Heavy Chain antibody, and observation by confocal microscopy, seeded on type I collagen.

FIG. 6C represents fluorescent immunostaining of smooth muscle cells with an anti-Calponin antibody, and observation by confocal microscopy, seeded on type I collagen.

FIG. 6D represents fluorescent immunostaining of smooth muscle cells with an anti-α-Smooth Muscle Actin antibody, and observation by confocal microscopy, seeded on PEMs.

FIG. 6E represents fluorescent immunostaining of smooth muscle cells with an anti-Smooth Muscle Myosin Heavy Chain antibody, and observation by confocal microscopy, seeded on PEMs.

FIG. 6F represents fluorescent immunostaining of smooth muscle cells with an anti-Calponin antibody, and observation by confocal microscopy, seeded on PEMs.

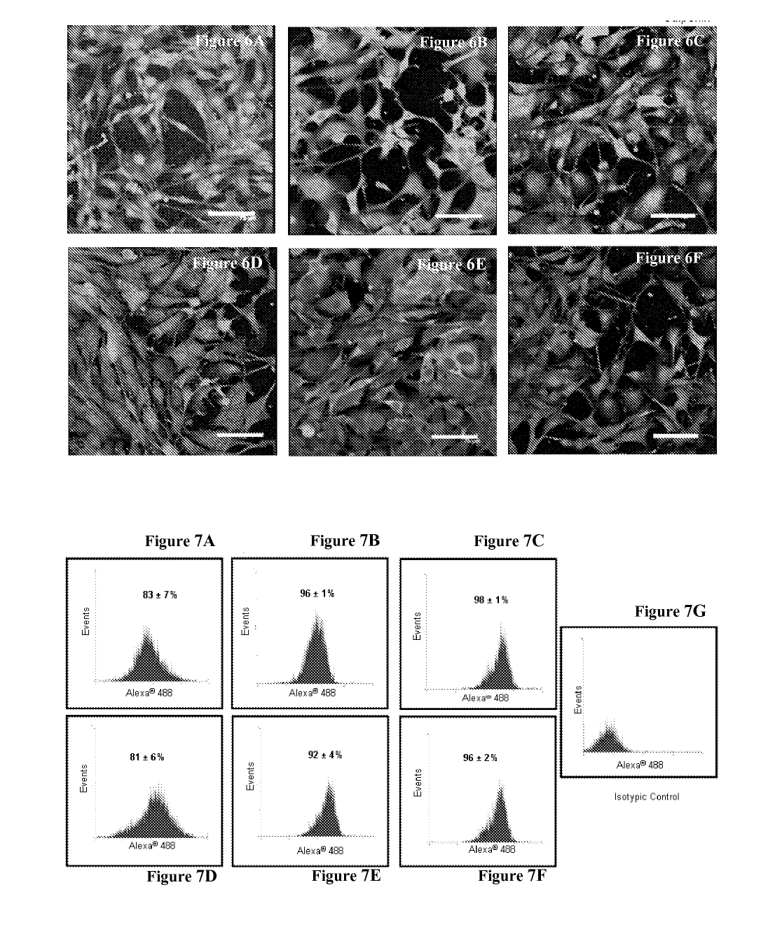

FIGS. 7A-G represent Flow cytometry analysis of cells labeled with anti SMCs markers antibodies coupled with Alexa®488 fluorochrome.

FIG. 7A shows that 83±7% of cells seeded on type I collagen express α-Smooth Muscle Actin.

FIG. 7B shows that 96±1% of cells seeded on type I collagen express Smooth Muscle Myosin Heavy Chain.

FIG. 7C shows that 83±7% of cells seeded on type I collagen express Calponine.

FIG. 7D shows that 83±7% of cells seeded on PEMs express α-Smooth Muscle Actin.

FIG. 7E shows that 83±7% of cells seeded on PEMs express Smooth Muscle Myosin Heavy Chain.

FIG. 7F shows that 83±7% of cells seeded on PEMs express Calponin.

FIG. 7G shows the result obtained with a control isotype antibody.

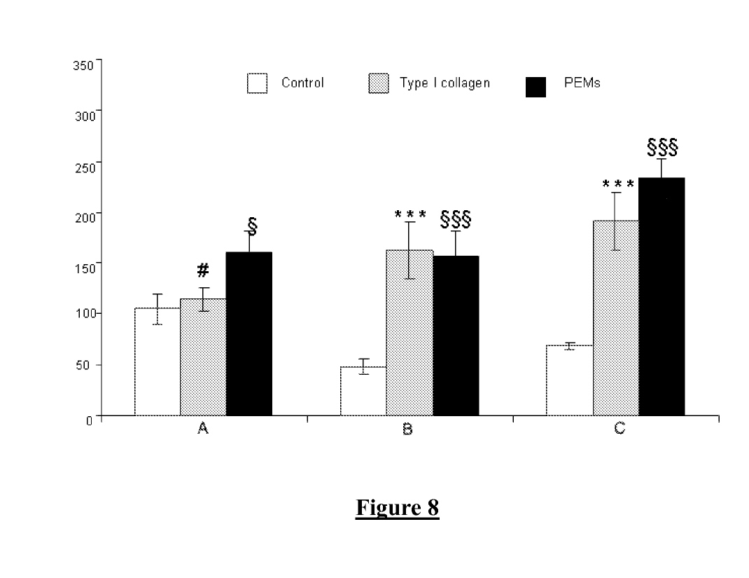

FIG. 8 represents the mean fluorescence intensity of analyses with SMCs contractile markers antibodies compared to control (mature SMCs). White columns represent cells seeded on control support, Grey columns represents cells seeded on type I collagen, Black columns represent cells seeded on PEMs. A represents cells labelled with an anti α-SMA antibody, B represents cells labelled with an anti SMMHC antibody and C represents cells labelled with an anti Calponine antibody.

(§) PEMs versus control, (*) Collagen versus control, (#) PEMs versus collagen. (§,* and #: p<0.05 and §§§ and ***: p<0.001).

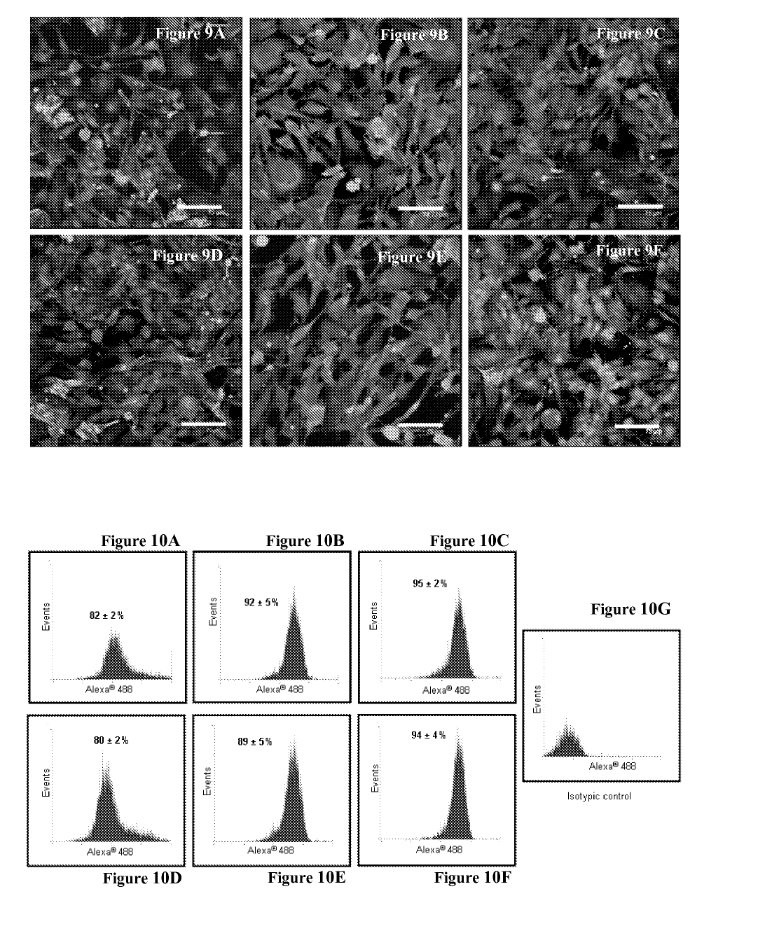

FIGS. 9A-F represent the phenotype stability under normoxia analysed by confocal microscopy after immunostaining with contractile markers α-Smooth Muscle Actin (α-SMA), Smooth Muscle Myosin Heavy Chain (SM-MHC) and Calponin antibodies on both coated surfaces (type I collagen and Polyelectrolyte Multilayer films (PEMs)). Objective×40, NA=0.8, scale bars 75 μm.

FIG. 9A represents fluorescent immunostaining of smooth muscle cells with an anti-α-Smooth Muscle Actin antibody, and observation by confocal microscopy, seeded on type I collagen.

FIG. 9B represents fluorescent immunostaining of smooth muscle cells with an anti-Smooth Muscle Myosin Heavy Chain antibody, and observation by confocal microscopy, seeded on type I collagen.

FIG. 9C represents fluorescent immunostaining of smooth muscle cells with an anti-Calponin antibody, and observation by confocal microscopy, seeded on type I collagen.

FIG. 9D represents fluorescent immunostaining of smooth muscle cells with an anti-α-Smooth Muscle Actin antibody, and observation by confocal microscopy, seeded on PEMs.

FIG. 9E represents fluorescent immunostaining of smooth muscle cells with an anti-Smooth Muscle Myosin Heavy Chain antibody, and observation by confocal microscopy, seeded on PEMs.

FIG. 9F represents fluorescent immunostaining of smooth muscle cells with an anti-Calponin antibody, and observation by confocal microscopy, seeded on PEMs.

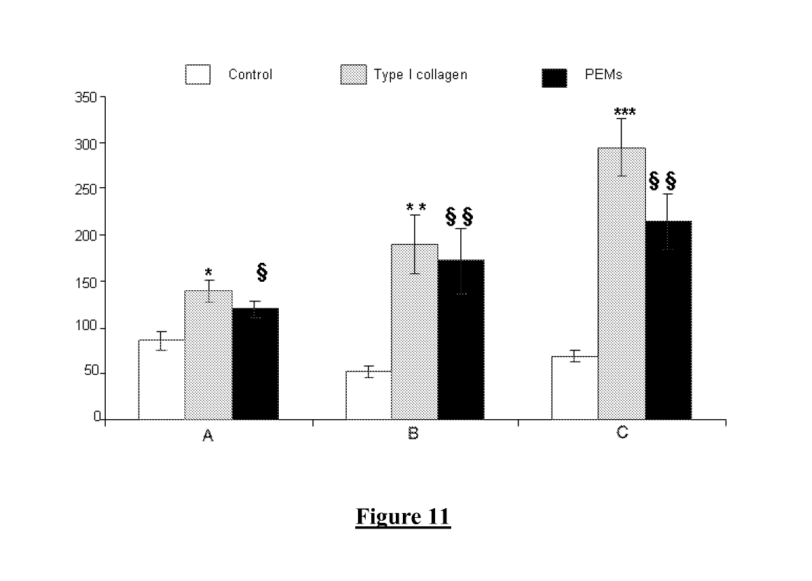

FIGS. 10A-G represent Flow cytometry analysis of cells labeled with anti SMCs markers antibodies coupled with Alexa®488 fluorochrome.

FIG. 10A shows that 82±2% of cells seeded on type I collagen express α-Smooth Muscle Actin.

FIG. 10B shows that 92±5% of cells seeded on type I collagen express Smooth Muscle Myosin Heavy Chain.

FIG. 10C shows that 95±2% of cells seeded on type I collagen express Calponine.

FIG. 10D shows that 80±2% of cells seeded on PEMs express α-Smooth Muscle Actin.

FIG. 10E shows that 89±5% of cells seeded on PEMs express Smooth Muscle Myosin Heavy Chain.

FIG. 10F shows that 94±4% of cells seeded on PEMs express Calponin.

FIG. 10G shows the result obtained with a control isotype antibody.

FIG. 11 represents the mean fluorescence intensity of analyses with SMCs contractile markers antibodies compared to control (mature SMCs). White columns represent cells seeded on control support, Grey columns represents cells seeded on type I collagen, Black columns represent cells seeded on PEMs. A represents cells labelled with an anti α-SMA antibody, B represents cells labelled with an anti SMMH antibody and C represents cells labelled with an anti Calponine antibody.

(§) PEMs versus control, (*) Collagen versus control. (§ and *: p<0.05, §§ and **: p<0.01, and *** p<0.001).

### EXAMPLES

### Example 1

### O₂ Content: the Determinant Regulator of Progenitor Cells Differentiation into Endothelial or Smooth Muscle Cells

During embryogenesis, vasculogenesis is one of the first initiated processes. Conversely in the adult, the new vessels formation is initiated from the existent blood vessel ramifications. Data accumulated in recent years indicate that the circulating mononuclear cell (MNCs) fractions contain a population of bone marrow derived cells called progenitor cells that contribute to the neovascularization of injured vessels. Different authors [Asahara T, et al. (1997) Science 275: 964-967; Simper D, et al. (2002) Circulation 106: 1199-1204; Xie S Z, et al. (2008) J Zhejiang Univ Sci B 9: 923-930; Liu J Y, et al. (2007) Cardiovasc Res 75: 618-628 and Yeh E T, et al. (2003) Circulation 108: 2070-2073] suggested that these progenitor cells could differentiate in the presence of different specific cytokines and angiogenic growth factors (vascular endothelial growth factor (VEGF), platelet derived growth factor BB (PDGF-BB).), into mature and functional endothelial (ECs) or vascular smooth muscle (SMCs) cells depending on the added specific growth factors. During wound healing, ischemia, vascular wall remodelling or tumour development, the formation of new blood vessels is preceded by the recruitment of MNCs at the injured sites which further promote vasculogenesis [Takahashi T, et al. (1999) Nat Med 5: 434-438; Davie N J, et al. (2004) Am J Physiol Lung Cell Mol Physiol 286: L668-L678; Stenmark K R, et al. (2006) Circ Res 99: 675-691 and Kerdjoudj H, et al. (2008) J Am Coll Cardiol 52: 1589-1597]. Various authors investigated also the role of the oxygen concentration on stem cells differentiation and it was shown that hypoxia increased the production of angiogenic growth factors such as transforming growth factor β1, PDGF-BB and VEGF [Falanga V, et al. (1991) J Invest Dermatol 97: 634-637; Payne T R, et al. (2007) J Am Coll Cardiol 50: 1677-1684 and Cramer T, et al. (2004) Osteoarthritis Cartilage 12: 433-439.]. The main physiological factors implicated in cell differentiation are angiogenic growth factors (i.e: VEGF, bFGF and IGF) [Simper D, et al. (2002) Circulation 106: 1199-1204; Xie S Z, et al. (2008) J Zhejiang Univ Sci B 9: 923-930 and Conway E M, et al. (2001) Cardiovasc Res 49: 507-521] and a decrease of the oxygen level in the tissue (hypoxia) [Yeh E T, et al. (2003) Circulation 108: 2070-2073]. Oxygen plays a main role in physiological and pathological states [Grayson W L, et al. (2006) J Cell Physiol 207: 331-339]; it is a potent biochemical signalling molecule with important regulation properties for cellular behaviour (migration, differentiation, proliferation . . . ) [Malda J, et al. (2007) Tissue Eng 13: 2153-2162; Simon M C and Keith B (2008) Nat Rev Mol Cell Biol 9: 285-96 and Gerasimovskaya E V, et al. (2008) Angiogenesis 11: 169-182]. However, the possible involvement of hypoxia in MNCs differentiation into SMCs has never been demonstrated and even mentioned up to now.

The Inventors hypothesized here that the only oxygen concentration tuning combined with growth factors favouring ECs differentiation (VEGF, FGF, EGF, IGF) [Griese D P, et al. (2003) Circulation 108: 2710-2715] allow the differentiation of circulating progenitor cells into mature ECs or contractile SMCs, characteristic of mature vascular cells found in vivo.

The Inventors demonstrate that progenitor cells isolated from rabbit fraction cultivated onto specifically coated solid substrates (either by type I collagen: a compound of the arterial wall and known as an ideal substrate for adhesion and proliferation of vascular smooth muscle cells in vitro [Simper D, et al. (2002) Circulation 106: 1199-1204] or by a Polyelectrolyte Multilayered Film architecture which previously demonstrated an important speeding up of endothelial progenitor cells differentiation into mature and functional endothelial cells [Berthelemy N, et al. (2008) Adv Mater 20: 2674-2678]) in normoxic conditions (21% O₂ atmosphere or 151 mmHg) lead to mature ECs and to SMCs when cultivated in exactly the same medium but under moderate hypoxic conditions (5% O₂ or 36 mmHg). Whereas it is well established that the culture of mature SMCs leads to a decrease of contractile markers associated with a pathological phenotype [Reusch P, et al. (1996) Circ Res 79: 1046-1053; Rovner A S, et al. (1986) J Biol Chem 261: 740-745 and Muto A, et al. (2007) J Vasc Surg 45: A15-24], the Inventors focused on SMCs-like cells obtained under hypoxia conditions and the Inventors checked the preservation of the contractile phenotype after further cell expansion (effect of passage number) and culture even under normoxic conditions.

These experiments demonstrate clearly the deterministic role of the oxygen content in vascular progenitor cells differentiation into mature functional cells constituting the vascular wall (media and intima).

### Methods

### 1) Polyelectrolyte Multilayer Films (PEMs)

PEMs were built with cationic poly (allylamine hydrochloride) (PAH, MW=70 kDa), and anionic poly(sodium-4-styrene sulfonate) (PSS, MW=70 kDa) solutions (Sigma-Aldrich, France) as previously described [19, 23]. Briefly, PEMs were prepared on glass coverslips (CML, Nemours, France) pretreated with 0.01 M SDS and 0.12 M HCl for 15 min at 100° C. and then extensively rinsed with deionized water. Glass coverslips were deposited in 24-well plates (Nunc, France). PAH-(PSS-PAH)₃ films were obtained by alternated immersion of the pretreated coverslips for 10 min in polyelectrolyte solutions (300 μL) at 5 mg/mL in the presence of 10 mM Tris-(hydroxymethyl)aminoethane (Tris) and 150 mM NaCl at pH 7.4. After each deposition, the coverslips were rinsed three times during 10 min with 10 mM Tris and 150 mM NaCl at pH 7.4. All the films were sterilized for 10 min by UV light (254 nm).

2) Isolation and Culture of Mononuclear Cells from Peripheral Blood Circulation.

The experimental procedures were used in accordance with the “Principle of Laboratory Animal Care and the Guide for the Care and Use of Laboratory Animals” (National Institute of Health publication No. 80-23, revised 1978). Blood (50 mL) was collected from white New Zealand rabbits (male, average weight 3-3.5 kg, CEGAV, France) carotid into heparinised plastic syringes. Peripheral Blood Mononuclear Cells (MNCs) were isolated using a density gradient as previously described [Berthelemy N, et al. (2008) Adv Mater 20: 2674-2678]. The cells were then cultivated in endothelial basal medium (EBM-2: Lonza, Belgium) supplemented with angiogenic growth factors (EGM-2-singleQuots® Lonza, Belgium). Cells were counted using Trypan Blue® and were seeded at a density of 1×10⁶ cells/cm² in 24-well plates containing glass coverslips coated either by Type I collagen 1% (BD Biosciences, France) or a PEMs films, made of PSS and PAH (Sigma, France) with a final PAH-(PSS-PAH)₃ architecture corresponding to 3.5 pairs of deposited PAH/PSS layers [Berthelemy N, et al. (2008) Adv Mater 20: 2674-2678]. The cultures were placed in normal cell culture incubator at 37° C. in an atmosphere with 5% CO₂ and 21% O₂, (O₂/CO₂ incubator, Sanyo, France). After three days, the medium was removed in order to discard unattached cells. The cells (CD34⁺, CD133⁺ were identified previously [Berthelemy N, et al. (2008) Adv Mater 20: 2674-2678]) were then placed under hypoxia at 37° C., 5% CO₂ and 5% O₂ or under normoxia at 37° C., 5% CO₂ and 21% O₂ (control) and medium changed every two days. The differentiation and morphological evolution of the adherent cells were followed by Phase-contrast microscopy observations (Nikon DIAPHOT 300, Japan).

### 3) Immunostaining for Smooth Muscle Cells (SMCs) and Endothelial Cells (ECs) Specific Markers

At confluence and after the third passage, cells were also immunolabelled against SMCs and ECs specific markers. Three antibodies were used to characterize the contractile SMCs phenotype: i) Alpha Smooth Muscle Actin (α-SMA), ii) Smooth Muscle Myosin Heavy Chain (SM-MHC) and iii) Calponin. Two other antibodies were used for the ECs phenotype: i) CD31 ii) von Willebrand factor (vWF) (all from Dako, France). Prior to the immunolabelling with the intracellular antibodies (α-SMA, SM-MHC, Calponin and vWF), the cells were fixed with paraformaldehyde (PAF) 4% (w/v in phosphate buffer saline) for 10 min and permeabilized with Triton X-100 0.5% (w/v in distilled water) for 15 min. For CD31 labelling the second step (permeabilization) was not performed. The cells were incubated for 45 min at 37° C. with the primary monoclonal antibodies, diluted at 1/50 in RPMI 1640 without phenol red, containing bovine serum albumin (BSA 0.5%, w/v). After two washes with RPMI 1640, the secondary antibody labelled with Alexa-Fluor® 488 diluted at 1/100 was incubated for 30 min at 37° C. The cells were observed by fluorescence confocal microscopy (LEICA DMIRE2 HC Fluo TCS 1-B, Germany) using the 488 nm spectral line.

### 4) Immunostaining for Extracellular Matrix (ECM) Proteins

At confluence, hypoxia differentiated cells were immunostained for ECM proteins characterization via two specific proteins such as i) laminin and ii) type IV collagen. The differentiated cells were fixed with PAF 4% for 10 min and incubated for 45 min at 37° C. with the primary monoclonal antibodies, diluted at 1/50 in RPMI 1640 without phenol red, containing 0.5% BSA. After two washes with RPMI 1640, the secondary antibody labelled with Alexa-Fluor® 488 diluted at 1/100 was incubated for 30 min at 37° C. The cells were observed using fluorescence confocal microscopy (LEICA DMIRE2 HC Fluo TCS 1-B, Germany).

### 5) Evaluation of the Maintenance of the SMCs Phenotype

In order to check that after a first step of culture under hypoxia, the differentiation into SMCs was stable versus time, cells were further cultivated either under hypoxia or normoxia. After differentiation the confluent cells cultivated on type I collagen and PEMs were amplified and separated in two batches. The first batch was kept under hypoxic condition (37° C., 5% CO₂ and 5% O₂) whereas the second batch was placed in normoxic conditions (37° C., 5% CO₂ and 21% O₂). Cells were then cultivated in these different conditions until the third passage (P3) and mature SMCs from rabbit aorta cultivated under the same conditions were used as control.

### 6) Fluorescence Activated Cell Sorting (FACS)

FACS analyses (EPICS XL, Beckman Coulter, France) were performed to quantify the percentage of positive cells and the fluorescence intensity of the specific contractile markers expressed by the differentiated SMCs. After P3, FACS was performed to identify intracellular antigens in cells. For that, trypsinized differentiated cells were labelled as previously described. The non-specific binding was evaluated by the incubation of cells only with the second antibody. Within the differentiated cell area, as determined by forward and sideward scattering, 10,000 events were collected and the percentage of positive cells and the mean fluorescence intensity (MFI) were determined.

### 7) Statistics

The data were expressed as mean±standard error of the mean (s.e.m.) for each condition. Each experiment was repeated in triplicate independently three times. Mean values were compared with the unpaired t-test (Statview IV, Abacus Concepts Inc, Berkley, Calif., USA), in which p represents the rejection level of the null-hypothesis of equal means.

### Results and Discussion

The following results are obtained with peripheral blood mononuclear cells. Similar results were obtained with MNC isolated from bone marrow, adipose tissues, umbilical cord blood or Wharton's jelly (data not shown).

Peripheral blood mononuclear cells (MNCs) fraction containing progenitor cells was isolated and seeded in 24-well plates containing glass coverslips coated with type I collagen or with a Polyelectrolyte Multilayer Film (PEMs) at 1×10⁶ cells/cm². The Inventors used type I collagen known as an ideal substrate for vascular progenitor cells culture [Simper D, et al. (2002) Circulation 106: 1199-1204] and PEMs for their high potentialities to boost progenitor cell differentiation [Berthelemy N, et al. (2008) Adv Mater 20: 2674-2678]. After 4 days of culture in normoxic conditions, unattached cells were removed and the adherent cells (CD34⁺, CD133⁺) were divided in two fractions and placed under hypoxia (5% CO₂ and 5% O₂) or normoxia (5% CO₂ and 21% O₂) until confluence (between 2 and 4 weeks). At confluence and for both surface types, the phase-contrast microscopy cell observation showed cobblestone morphology in normoxic conditions (FIG. 1A, 1C) and a spindle like morphology in hypoxic conditions (FIG. 1B, 1D).

In order to evaluate the cell phenotype of differentiated cells, the Inventors checked the expression of specific markers of vascular cells (SMCs and ECs) i.e. alpha-Smooth Muscle Actin (α-SMA), Smooth Muscle Myosin Heavy Chain (SM-MHC) and Calponin known to assess vascular SMCs differentiation and their contractile function [Simper D, et al. (2002) Circulation 106: 1199-1204; Babu et al. (2004) Am J Physiol Cell Physiol 287: 723-729 and Li S, et al. (2001) Circ Res 89: 517-525] and CD31 and von Willebrand Factor (vWF) for the ECs phenotype evaluation [Newman P J, et al. (1990) Science 247: 1219-1222 and Meyer D, at al. (1991) Mayo Clin Proc 66: 516-523]. As expected under normoxic conditions, the confocal microscopy observations showed the presence of positive cells for ECs markers [FIGS. 1E and 1I (for type I collagen coating), 1G and 1K (for PEMs coating)] and negative cells for SMCs markers [FIGS. 1M, 1Q and 1U (for type I collagen), 1O, 1S and 1W (for PEMs)]. Under hypoxia a surprising positive expression of SMCs markers was observed [FIGS. 1N, 1R and 1V (for type I collagen), 1P, 1T and 1X (for PEMs)]. No expression of ECs markers was noticed under this condition whatever the surface coating [FIGS. 1F and 1J (for Type I collagen), FIGS. 1H and 1L (for PEMs)] indicating thus a total absence of cellular differentiation into ECs at a low concentration of O₂. All these observations constitute a signature for the progenitor cells switching into SMCs phenotype. These results suggest first the potentiality of MNCs cells to differentiate into a SMCs phenotype under a hypoxic environment and second the expression of the specific markers confirmed the contractile phenotype of these cells [Owens G K (1995) Physiol Rev 75: 487-517] (similar to SMCs in vivo). In the literature the hematopoietic stem cells differentiation into mature and functional SMCs requires the culture medium supplementation with specific growth factors, especially PDGF-BB [Simper D, et al. (2002) Circulation 106: 1199-1204 and Xie S Z, et al. (2008) J Zhejiang Univ Sci B 9: 923-930]. Our results demonstrate that the oxygen concentration tuning alone allows phenotype switch either to endothelial cells or smooth muscle cells.

The extracellular matrix (ECM) contributes to the control of the cellular function and is involved in maintaining the cells in a differentiated state [Ingber D E, et al. (1994) Int Rev Cytol 150:173-224 and Bissell M J and Barcellos-Hoff M H (1987) J Cell Sci 8: 327-343]. During blood vessel formation the SMCs are responsible for extracellular matrix formation via protein (fibronectin, laminin, collagens . . . ) secretion [Rzucidlo E M, et al. (2007) J Vasc Surg 45: 25-32]. The ECM deposition contributes in vivo and in vitro (tissue engineering approach) to arterial wall constitution and cell function via different signalling pathways (kinase pathways activation) [Rzucidlo E M, et al. (2007) J Vasc Surg 45: 25-32 and Davis M J, et al. (2001) Am J Physiol Heart Circ Physiol. 280: H1427-H1433]. The Inventors investigated the capacity of the differentiated cells under hypoxic conditions to synthesize their own ECM, and the Inventors evaluated the secretion of two extracellular proteins (Laminin and type IV collagen), which play a major role in ECM synthesis and contribute to maintain the contractile phenotype of the differentiated cells [Rzucidlo E M, et al. (2007) J Vasc Surg 45: 25-32]. Confocal microscopy observations showed the deposition of both of these proteins. The comparison between both surfaces showed moreover a stronger synthesis of ECM by the cells cultivated on PEMs (FIG. 2A-D). These data obtained under hypoxic conditions confirmed the capacity of MNCs to differentiate into SMCs, exhibiting a contractile phenotype, sign of a correct physiological state and integrity of the ECM. This integrity plays a key role to maintain this state and suggests stability over longer time periods. The phenotype stability over a longer time period of the SMCs derived from MNCs cultivated under hypoxia is a major issue to use this route in tissue engineering for example. The SMCs phenotype stability was investigated at low or high oxygen concentration. After the first passage of hypoxic differentiated cells (cells positive to SMCs markers), the obtained cells were expanded under two conditions. For the first assay the Inventors maintained cells under hypoxic condition and for the second assay the Inventors placed cells in normoxic condition. In order to check the stability of the SMCs phenotype under these conditions, several passages (P3) were performed. Whatever the experimental condition (hypoxic and normoxic conditions) the Inventors never detected ECs markers (data not shown).

Under hypoxia the cell characterization showed the positive staining for SMCs markers with a regular cytosolic distribution of all observed SMCs markers (FIG. 6A-F) for both coating types (Type I collagen and PEMs). These data were correlated with FACS analyses which indicated that, after the third passage, more than 80% of cells were positive for both surfaces (FIG. 7A-G). The Inventors compared moreover the Mean Fluorescence Intensity (MFI) of SMCs contractile markers expression of the differentiated cells with mature SMCs extracted from rabbit aorta and cultivated in the same medium in normoxic and hypoxic conditions. Mature SMCs were cultivated on the usually employed tissue culture plastic surface (TCPS) [L'Heureux N, et al. (2001) FASEB J 15: 515-24] showing no difference with a control performed on type I collagen and PEMs. The expression of α-SMA, SM-MHC and calponin for cells cultivated on both Type I collagen and PEM coated surfaces was significatively higher for the differentiated cells compared to mature SMCs, although less important on the collagen coated surface for α-SMA (FIG. 8).

Under normoxic conditions, the expanded cells were also qualitatively and quantitatively characterized by confocal microscopy observations and by FACS analyses. As for hypoxic conditions, the visualized cells were positive for SMCs contractile markers with again a regular cytoplasmic distribution (FIG. 9A-F). FACS analyses showed also that more than 80% of differentiated cells were positive to SMCs contractile markers (FIG. 10A-G). The MFI of contractile markers for differentiated cells was significatively higher than for mature SMCs for both surfaces coating and with no differences for differentiated cells cultivated on type I collagen and PEMs coated surfaces (FIG. 11). It is also important to state that no significatively difference was found in the expression of the three contractile markers once comparing the data obtained in hypoxic and normoxic conditions.

It is well known that in vitro mature SMCs extracted from vessels switch their phenotype from a contractile (healthy) to a proliferative (pathological) phenotype [Cha J M, et al. (2005) Artif Organs 30: 250-258 and Bach A D, et al. (2003) Clin Plast Surg 30: 589-599]. This switch constitutes a strong limitation for blood vessel tissue engineering. The present differentiation approach allowed us to obtain a “healthy” phenotype of SMCs which could constitute an alternative for vascular tissue engineering. The Inventors observed effectively a quite stronger expression of the contractile markers for the differentiated cells compared to mature SMCs. In vivo, after vascular injuries, the inflammatory reactions, involving MNC, are implicated in the healing process. Thus the vascular wall remodeling after rabbit carotid bypass was investigated. An antithrombogenic graft with suitable mechanical properties was implanted [Kerdjoudj, H. et al. Adv. Funct. Mater. 17, 2667-2673 (2007); Kerdjoudj H, et al. (2008) J Am Coll Cardiol 52: 1589-1597]. The wall graft behaviour was followed until 12 weeks. Less than one month after implantation, histological analysis revealed graft wall necrosis leading to a total loss of vascular cells (SMC) due to absence of vasa vasorum (responsible of vessel vascularization) [L'Heureux, N. et al. Nat Med. 12, 361-365 (2006)] and the presence of inflammatory cells surrounding the vessel (FIG. 3). Twelve weeks after implantation, strong differences in the wall structure appeared as compared to the previous observations. Beside their ability to remain permeable to blood flow, the histological observations showed i) a total resorption of the inflammatory cells, and ii) the vascular wall recolonization. The cell identification demonstrated the presence of positive α-SMA cells signature for the SMC phenotype. It has been showed[L'Heureux, N. et al. Nat Med. 12, 361-365 (2006)] that the formation of vasa vasorum after one month of implantation allowing oxygen access (2% to 9% concentration range comparable to in vitro hypoxic condition).

Moreover, the H&S staining showed the predominance of collagen in adventitia and elastin in media. The same observations were made by several vascular tissue engineering studies without demonstrating the origin of SMC [Mellander, S., et al. (2005) Eur J Vasc Endovasc Surg. 30, 63-70; L'Heureux, N. et al. (2006) Nat Med. 12, 361-365; Chaouat, M., et al. (2006) Biomaterials 27, 5546-53]. The present data highlight the role of the inflammatory cells in the healing process which combined with the low oxygen level in the vascular wall participates in the vascular wall remodelling.

To conclude the Inventors demonstrated that progenitor cells cultivated in hypoxic conditions and without specific growth factor enhancing SMCs differentiation displayed morphological and phenotypic properties of SMCs as showed by the expression of SMCs contractile markers. Moreover, these differentiated SMCs maintained their contractile phenotype when replaced in normoxic conditions suggesting that these cells developed a stable and functional phenotype comparable to physiological SMCs found in functional blood vessels.

These results highlight the crucial role of the tissue environment and especially the O₂ content in the differentiation process of vascular progenitor cells. These observations combined with previous ones [Berthelemy N, et al. (2008) Adv Mater 20: 2674-2678] could constitute a basis for tissue engineering and clinical application strategies for in vitro tissue reconstruction. For example in vascular tissue engineering, starting from an unique peripherical blood sample cultivated on PEM and with the same culture media, but in normoxic or in hypoxic conditions either mature ECs (21% O₂) or contractile SMCs (5% O₂) can be obtained in less than one month. The different layers (media and intima) could be associated to build for example a natural a natural and autologous vascular graft.

### Example 2

### Functional Blood Vessel Construction from Hematopoietic Stem Cells Differentiation

The present example discloses an example of protocol for building an in vitro blood vessel, according to the process of the invention. This example is illustrated by FIG. 4 and FIG. 5. Hereafter, “mononucleated” cells refers to normal cells that contain a nucleus. Thus, red blood cells, apoptotic cells, and cellular fragments, etc. . . . are excluded of this definition. Mononucleated cells are therefore stem cells and differentiated cells.

### Matrix Preparation (Support).

First, the support is built as mentioned above, and deposited on an appropriate surface. Cells are deposited on support.

The removal of differentiated cells from support can be achieved by varying ionic force (ion concentration), temperature or pH, or any methods known in the art to allow the recovery of functional living cells.

### Cell Differentiation (FIG. 4).

Hematopietic stem cells and mesenchymatous stem cells can be used in this process. These stem cells can be purified from:
blood (B),
bone marrow (BM),
Warthon jelly (WJ)
Umbilical cord blood (UCB), or
Adipose tissues (AT).

The following protocols illustrate processes for purifying the above mentioned stem cells. These protocols can be easily modified by a skilled person, in particular by modifying serum concentration, according to the manufacturer instructions.

Cell Preparation from Blood

Blood was removed from individual, and placed into a centrifugation tube containing a density gradient (a) (for example: Histopaque 1077 for rabbits cells, Lymphoprep for human cells). After centrifugation (500 g, 10 min), mononucleated cells were separated from the pellet containing red blood cells (b).

Isolated mononucleated cells were then placed on a surface (c), covered by a support, in an appropriate culture medium [endothelial basal medium EBM-2 (Clonetics, Belgium)] supplemented with 5% serum and comprising growth factor (VEGF, R³-IGF, rhFGFb, ascorbic acid, rhEGF, heparin, Hydrocortison).

Cells were left in the culture medium for 4 days, to allow cell attachment (d1 and d2). Unseeded cells were then removed (e1 and e2) and seeded cells were placed in an appropriate O₂ containing atmosphere, i.e. in an atmosphere comprising a low concentration of oxygen (5%, hypoxia, f1) or in an atmosphere comprising a normal concentration of oxygen (20%, normoxia, f2).

Cell Preparation from Bone Marrow

Bone marrow was obtained by a ponction from a large bone of the donor, typically the pelvis, through a large needle that reaches the center of the bone. Bone marrow cells were placed into a centrifugation tube (a) and
either centrifugated (500 g, 10 min) to pellet mononucleated cells containing stem cells,
or by using cytapheresis procedure in order to collect mononucleated cells isolated from red blood cells.

Isolated mononucleated cells were then placed on a surface (c), covered by a support, in an appropriate culture medium (aMEM (Lonza) supplemented with 10% serum, Fungizone (Gibco, France) 2.5 μg/mL, Penicillin 50 UI/mL+Streptomycin (Gibco, France) 50 μg/mL, L-Glutamine (Gibco, France) 5 mM and FGF2 (R&D systems) 0.6 ng/mL).

Cells were left in the culture medium for 2 days, to allow cell attachment (d1 and d2). Unseeded cells were then removed (e1 and e2) and seeded cells were placed in an appropriate O₂ containing atmosphere, i.e. in an atmosphere comprising a low concentration of oxygen (5%, hypoxia, f1) or in an atmosphere comprising a normal concentration of oxygen (20%, normoxia, f2).

Cell Preparation from Umbilical Cord Blood

Umbilical cord blood was removed from post natal umbilical cord from a consenting mother, and placed into a centrifugation tube containing a density gradient (a) (for instance: Histopaque 1077, Lymphoprep for human cells). After centrifugation (450 g, 30 min, 25° C.), mononucleated cells were separated from the pellet containing red blood cells (b).

Isolated mononucleated cells were then placed on a surface (c), covered by a support, in an appropriate culture medium [endothelial basal medium EBM-2 (Clonetics, Belgium)] supplemented with 5% serum and comprising growth factor (VEGF, R³-IGF, rhFGFb, ascorbic acid, rhEGF, heparin, Hydrocortison).

Cells were left in the culture medium for 7 days, to allow cell attachment (d1 and d2). Unseeded cells were then removed (e1 and e2) and seeded cells were placed in an appropriate O₂ containing atmosphere, i.e. in an atmosphere comprising a low concentration of oxygen (5%, hypoxia, f1) or in an atmosphere comprising a normal concentration of oxygen (20%, normoxia, f2).

Cell Preparation from Wharton Jelly

Umbilical cord was removed from post natal umbilical cord from a consenting mother, and placed into appropriate culture medium (aMEM (Lonza) supplemented with 10% serum, Fungizone (Gibco, France) 2.5 μg/mL, Penicillin 50 UI/mL+Streptomycin (Gibco, France) 50 μg/mL, L-Glutamine (Gibco, France) 5 mM and FGF2 (R&D systems) 0.6 ng/mL) (a). Vein and artery are removed and the umbilical cord was minced and the cells resulting from the dissociation of Wharton jelly were then placed on a surface (c), covered by a support, in an appropriate culture medium (aMEM (Lonza) supplemented with 10% serum, Fungizone (Gibco, France) 2.5 μg/mL, Penicillin 50 UI/mL+Streptomycin (Gibco, France) 50 μg/mL, L-Glutamine (Gibco, France) 5 mM and FGF2 (R&D systems) 0.6 ng/mL) (b).

Cells were left in the culture medium for 7 days, to allow cell attachment (d1 and d2). Unseeded cells were then removed by washing (e1 and e2) and seeded cells were placed in an appropriate O₂ containing atmosphere, i.e. in an atmosphere comprising a low concentration of oxygen (5%, hypoxia, f1) or in an atmosphere comprising a normal concentration of oxygen (20%, normoxia, f2).

Cell Preparation from Adipose Tissue (see also Locke et al. ANZ J Surg 79 (2009) 235-244).

Fat tissue was obtained from a lipoaspiration of an individual for instance and placed in a centrifugation tube (a). Residual red blood cells are lysed by a standard procedure (for instance Tris 10 mM/MgCl₂ 10 mM/NaCl 10 mM, or NH₄CO₃H 0.9 mM/NH₄Cl 131 mM, or Tris 20 mM pH7.5/MgCl₂ 5 mM or Tris 10 mM pH7.4/EDTA (ethylene diamine tetra-acetic acid) 10 mM for 20-30 min, 4° C.). Fat was digested by using collagenase. After centrifugation (450 g, 30 min, 25° C.), mononucleated cells contained in the lower phase were removed and placed on a surface (c), covered by a support, in an appropriate culture medium (aMEM (Lonza) supplemented with 10% serum, Fungizone (Gibco, France) 2.5 μg/mL, Penicillin 50 UI/mL+Streptomycin (Gibco, France) 50 μg/mL, L-Glutamine (Gibco, France) 5 mM and FGF2 (R&D systems) 0.6 ng/mL) (b).

Cells were left in the culture medium for 7 days, to allow cell attachment (d1 and d2). Unseeded cells were then removed by washing (e1 and e2) and seeded cells were placed in an appropriate O₂ containing atmosphere, i.e. in an atmosphere comprising a low concentration of oxygen (5%, hypoxia, f1) or in an atmosphere comprising a normal concentration of oxygen (20%, normoxia, f2).

Cells were then leaved in their culture medium, under their atmosphere for 14 days, for the achievement of cellular differentiation.

Cells that have grown under normoxic conditions are differentiated in endothelial cells, whereas cells that have grown under hypoxic conditions are differentiated in smooth muscle cells.

### Blood Vessel Building (FIG. 5).

Smooth muscle cells obtained from the previous step are then stimulated with growth factor such as ascorbic acid to enhance the density of the smooth muscle cells layer. This treatment allows the recovery of the take off the layer from the surface (pH variation).

Also, ionic variations and temperature variations can be used to take off the smooth muscle layer from the surface.

Then the smooth muscle cells layer is rolled up around a hydrophobic stake (for example composed by Teflon® (a & b).

The tube, rolled up around the stake, is placed in a bioreactor (generating shear and stretch) to induce the formation of a consolidated tube and to form a media (c).

Then, the stake is removed from the consolidated tube (d) and endothelial cells obtained from the previous step are added in the lumen of said tube (e).

The tube with endothelial cells is left for 1 week to allow the recovery of the lumen by a monolayer of endothelial cells, i.e. the intima (f).

The tube is then placed in a bioreactor (generating shear and stretch) to induce the formation of a consolidated tube and to allow the formation of an oriented intima (g).

Then a functional vessel is formed.

1-15. (canceled)

16. Process of differentiation of stem cells derived from bone marrow or blood or adipose tissue, or umbilical cord, = and seeded on a support, in an appropriate culture medium, wherein said differentiation leads to:
a first group of specialized differentiated cells under normoxic conditions, and in an appropriate culture medium, and
a second group of specialized differentiated cells under hypoxic conditions, in a culture medium of the same nature as the one used for obtaining the first group of specialized differentiated cells, wherein hypoxic conditions are different from anoxia,
said first and second groups of specialized differentiated cells retaining the functional properties of the corresponding specialized differentiated cells respectively obtained through a biological natural process,
the specialized differentiated cells of the first group having cellular functional properties different from the specialized differentiated cells of the second group,
said process using of specific oxygen concentrations for implementing an in vitro process

17. Process for the differentiation, of stem cells originating from bone marrow or blood or adipose tissue, or umbilical cord, seeded on a support, comprising the use of a binary set of two culture media with oxygen specific concentrations culture media, each oxygen specific concentrations culture medium corresponding to a culture medium with specific oxygen concentrations, said process allowing the differentiation respectively into:
a first group of specialized differentiated cells by culture of said stem cells on a support in a culture medium under normoxic conditions, and
a second group of specialized differentiated cells by culture of said stem cells on a support in a culture medium of the same nature as the one used for obtaining the first group of specialized differentiated cells, under hypoxic conditions, wherein hypoxic conditions are different from anoxia,
said first and second groups of specialized differentiated cells retaining the functional properties of the corresponding specialized differentiated cells respectively obtained through a biological natural process,
the specialized differentiated cells of the first group having cellular functional properties different from the specialized differentiated cells of the second group.

18. Process according to claim 16, wherein normoxic conditions are such that ambient air is constituted by oxygen concentrations comprised from 13% to 21% of molar content per volume (mc/v) of total ambient air gas, preferably from 15 to 20% of molar content per volume (mc/v) of total ambient air gas, and
wherein hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas.

19. Process according to claim 16, wherein the support comprises or is constituted by:
gelatin, fibronectin, collagen, laminin, RGD peptide, or association, or
polyelectrolyte multilayers, preferably polycations and polyanions, preferably alternate,
said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
and preferably chosen among (PAH-PSS)₃, (PAH-PSS)₃-PAH et PEI-(PSS-PAH)₃.
wherein the layer number of polyelectrolytes layers is from 1 to 100, preferably from 3 to 50, more preferably from 5 to 10, and in particular 7.
said support being deposited on a surface, preferably said surface is a natural or artificial surface, more preferably
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems,
said natural surface being chosen among blood vessels, veins, heart, small intestinal submucosa, arteries, preferably decellularised umbilical arteries, said vessels, veins, arteries originating from human organs.

20. Process according to claim 16, wherein said stem cells are chosen among mesenchymal stem cells (MSC) and hematopoietic stem cells (HSC).

21. Process according to claim 16, wherein the first and the second groups of specialized differentiated cells consist of cells chosen among endothelial cells and smooth muscle cells, and
wherein said first group of specialized differentiated cells consists of endothelial cells and said second group of specialized differentiated cells consists of smooth muscle cells.

22. Culture medium with oxygen specific concentrations comprising:
an appropriate culture medium, and
oxygen atmosphere concentrations in said culture medium comprised from 2% to 12% of molar content per volume (mc/v) of total air, preferably from 3 to 8% of molar content per volume (mc/v) of total air, and more preferably from 4 to 6% of molar content per volume (mc/v) of total air,
said culture medium with oxygen specific concentrations being preferably in association with a support deposited on a surface

23. Culture medium with oxygen specific concentrations comprising:
an appropriate culture medium,
oxygen at concentrations in said culture medium comprised from 13% to 21% of molar content per volume (mc/v) of total ambient air gas, preferably from 15 to 20% of molar content per volume (mc/v) of total ambient air gas,
in association with a support deposited on a surface.

24. Binary set of two culture media with oxygen specific concentration, each culture medium with oxygen specific concentration corresponding to an appropriate culture medium and specific oxygen concentrations, comprising:
an appropriate culture medium with oxygen at concentrations in said culture medium comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas, in association with a support deposited on a surface, and
an appropriate culture medium with oxygen at concentrations in said culture medium comprised from 13% to 21% of molar content per volume (mc/v) of total ambient air gas, in association with a support deposited on a surface.

25. Culture medium with oxygen specific concentrations according to claim 22, wherein said support deposited on a surface comprises or is constituted by:
gelatin, fibronectin, collagen, laminin, RGD peptide, or association, or
polyelectrolyte multilayers, preferably polycations and polyanions, preferably alternate,
said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
and preferably chosen among (PAH-PSS)₃, (PAH-PSS)₃-PAH et PEI-(PSS-PAH)₃.

26. Culture medium with oxygen specific concentrations according to claim 25, wherein said surface is a natural or artificial surface
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems or cultured system,
said natural surface being chosen among blood vessels, veins, heart, small intestine mucosa, arteries, preferably decellularised umbilical arteries, said vessels, veins, arteries derived from human organs.

27. Binary set of two culture media with oxygen specific concentration to according claim 24, wherein said support deposited on a surface comprises or is constituted by:
gelatin, fibronectin, collagen, laminin, RGD peptide, or association, or
polyelectrolyte multilayers, preferably polycations and polyanions, preferably alternate,
said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
and preferably chosen among (PAH-PSS)₃, (PAH-PSS)₃-PAH et PEI-(PSS-PAH)₃.

28. Process of differentiation of stem cells, derived from bone marrow or blood, or adipose tissue, or umbilical cord, provided that said stem cells are not human embryonic stem cells, and are preferably chosen among mesenchymatous stem cells (MSC) and hematopoietic stem cells (HSC) comprising:
contacting stem cells originating from bone marrow or blood, or adipose tissue, provided that said stem cells are not human embryonic stem cells, with a support deposited on a surface in an appropriate culture medium, to obtain seeded stem cells on a support,
varying oxygen concentrations in said appropriate culture medium containing said seeded stem cells on the support, to provide normoxic or hypoxic conditions, said hypoxic conditions being different from anoxia
leaving the achievement of the differentiation of said seeded stem cells on the support,
either into a first group of specialized differentiated cells by culture of said seeded stem cells on a support under normoxic conditions,
or into a second group of specialized differentiated cells by culture of said seeded stem cells on a support, in a culture medium of the same nature as the one used for obtaining the first group of specialized differentiated cells, under hypoxic conditions,
said first and second groups of specialized differentiated cells retaining the functional properties of the corresponding specialized differentiated cells respectively obtained through a biological natural process,
the specialized differentiated cells of the first group having cellular functional properties different from the specialized differentiated cells of the second group.

29. Process of functional blood vessel formation using a binary set of two culture media with oxygen specific concentration, each oxygen specific concentration culture medium corresponding to an appropriate culture medium with specific oxygen concentrations,
said process comprising the following steps:
contacting stem cells, preferably chosen among mesenchymatous stem cells (MSC) and hematopoietic stem cells (HSC), derived from bone marrow or blood, or adipose tissue, or umbilical cord, provided that said stem cells are not human embryonic stem cells, with a support deposited on a surface in an appropriate culture medium, to obtain seeded stem cells on a support,
varying oxygen concentrations in said appropriate culture medium containing seeded stem cells on a support, to provide normoxic or hypoxic conditions, said hypoxic conditions being different from anoxia
leaving the achievement of the = differentiation of said seeded stem cells on a support, respectively into:
a first group of specialized differentiated cells by culture of said seeded stem cells on a support in a culture medium under normoxic conditions, said first group of specialized differentiated cells preferably consists of endothelial cells, and
a second group of specialized differentiated cells by culture of said seeded stem cells on a support in a culture medium of the same nature as the one used for obtaining the first group of specialized differentiated cells, under hypoxic conditions, said second group of specialized differentiated cells preferably consists of smooth muscle cells,
collecting respectively the first and the second group of specialized differentiated cells, and
building-up a vessel constituted by a second group of specialized differentiated cells layer outside, and a first group of specialized differentiated cells monolayer inside, and limiting the lumen, and hence allowing the formation of a functional blood vessel.

30. Process according to claim 28, wherein
said normoxic conditions are such that ambient air is constituted by oxygen concentrations comprised from 13% to 21% of molar content per volume (mc/v) of total ambient air gas, preferably from 15 to 20% of molar content per volume (mc/v) of total ambient air gas, and
said hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas.

31. Process according to claim 28, wherein said support comprises or is constituted by:
gelatin, fibronectin, collagen, laminin, RGD peptide, or association, or
polyelectrolyte multilayers, preferably polycations and polyanions, preferably alternate,
said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
and preferably chosen among (PAH-PSS)₃, (PAH-PSS)₃-PAH et PEI-(PSS-PAH)₃.
said support being deposited on a surface,
said surface being preferably a natural or artificial surface, more preferably
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems,
said natural surface being chosen among blood vessels, veins, heart, small intestine mucosa, arteries, preferably decellularised umbilical arteries, said vessels, veins, arteries originating from human organs.

32. Process according to claim 17, wherein normoxic conditions are such that ambient air is constituted by oxygen concentrations comprised from 13% to 21% of molar content per volume (mc/v) of total ambient air gas, preferably from 15 to 20% of molar content per volume (mc/v) of total ambient air gas, and
wherein hypoxic conditions are such that ambient air is constituted by oxygen concentrations comprised from 2% to 12% of molar content per volume (mc/v) of total ambient air gas, preferably from 3 to 8% of molar content per volume (mc/v) of total ambient air gas, and more preferably from 4 to 6% of molar content per volume (mc/v) of total ambient air gas.

33. Process according to claim 17, wherein the support comprises or is constituted by:
gelatin, fibronectin, collagen, laminin, RGD peptide, or association, or
polyelectrolyte multilayers, preferably polycations and polyanions, preferably alternate,
said polycations being chosen among the group comprising: polyallylamine (PAH), polyethyleneimine (PEI), polyvinylamine, polyaminoamide (PAMAM), polyacrylamide (PAAm), polydiallyldimethylammonium chlorure (PDAC), positively charged polypeptides such as polylysine and polysaccharides negatively charged such as chitosane, and
said polyanions being chosen among the group comprising: polyacrylic acid (PAA), polymetacrylic acid (PMA), polystyrene sulfonic acid (PSS or SPS), negatively charged polypeptides such as polyglutamic acid and polyaspartic acid and polysaccharides negatively charged such as hyaluronan and alginate,
and preferably chosen among (PAH-PSS)₃, (PAH-PSS)₃-PAH et PEI-(PSS-PAH)₃.
wherein the layer number of polyelectrolytes layers is from 1 to 100, preferably from 3 to 50, more preferably from 5 to 10, and in particular 7.
said support being deposited on a surface, preferably said surface is a natural or artificial surface, more preferably
said artificial surface being chosen among glass, TCPS (polystyrene cell culture treated), polysiloxane, perfluoalkyle polyethers, biocompatible polymers, in particular Dacron®, polyurethane, polymethylsiloxane, polyvinyl chlorure, Silastic®, expanded polytetrafluoroethylene (ePTFE), and any material used for prothesis and/or implanted systems,
said natural surface being chosen among blood vessels, veins, heart, small intestinal submucosa, arteries, preferably decellularised umbilical arteries, said vessels, veins, arteries originating from human organs.

34. Process according to claim 17, wherein said stem cells are chosen among mesenchymal stem cells (MSC) and hematopoietic stem cells (HSC).

35. Process according to claim 17, wherein the first and the second groups of specialized differentiated cells consist of cells chosen among endothelial cells and smooth muscle cells, and
wherein said first group of specialized differentiated cells consists of endothelial cells and said second group of specialized differentiated cells consists of smooth muscle cells.
